SEQUENCE LISTING (77 sequences)

SEQ ID NO: 1 - protein, 10 aa, Artificial Sequence
  Synthetic
GDTFSTYAIS

SEQ ID NO: 2 - protein, 10 aa, Artificial Sequence
  Synthetic
  MISC_FEATURE (5)..(5): Xaa is Ile or Leu
GIIPXFGKAH

SEQ ID NO: 3 - protein, 17 aa, Artificial Sequence
  Synthetic
GIIPIFGKAHYAQKFQG

SEQ ID NO: 4 - protein, 17 aa, Artificial Sequence
  Synthetic
GIIPLFGKAHYAQKFQG

SEQ ID NO: 5 - protein, 14 aa, Artificial Sequence
  Synthetic
  MISC_FEATURE (3)..(3): Xaa is His or Arg
  MISC_FEATURE (6)..(6): Xaa is Ser or Arg
KFXFVXGSPFGMDV

SEQ ID NO: 6 - protein, 14 aa, Artificial Sequence
  Synthetic
KFHFVSGSPFGMDV

SEQ ID NO: 7 - protein, 14 aa, Artificial Sequence
  Synthetic
KFHFVRGSPFGMDV

SEQ ID NO: 8 - protein, 14 aa, Artificial Sequence
  Synthetic
KFRFVSGSPFGMDV

SEQ ID NO: 9 - protein, 11 aa, Artificial Sequence
  Synthetic
  MISC_FEATURE (10)..(10): Xaa is Leu or Met
  MISC_FEATURE (11)..(11): Xaa is Ala, Ser, or Glu
RASQSVSSYXX

SEQ ID NO: 10 - protein, 11 aa, Artificial Sequence
  Synthetic
RASQSVSSYLA

SEQ ID NO: 11 - protein, 11 aa, Artificial Sequence
  Synthetic
RASQSVSSYLS

SEQ ID NO: 12 - protein, 11 aa, Artificial Sequence
  Synthetic
RASQSVSSYMA

SEQ ID NO: 13 - protein, 11 aa, Artificial Sequence
  Synthetic
RASQSVSSYLE

SEQ ID NO: 14 - protein, 7 aa, Artificial Sequence
  Synthetic
  MISC_FEATURE (7)..(7): Xaa is Thr, Pro, Met, or Glu
DASNRAX

SEQ ID NO: 15 - protein, 7 aa, Artificial Sequence
  Synthetic
DASNRAT

SEQ ID NO: 16 - protein, 7 aa, Artificial Sequence
  Synthetic
DASNRAP

SEQ ID NO: 17 - protein, 7 aa, Artificial Sequence
  Synthetic
DASNRAM

SEQ ID NO: 18 - protein, 7 aa, Artificial Sequence
  Synthetic
DASNRAE

SEQ ID NO: 19 - protein, 8 aa, Artificial Sequence
  Synthetic
  MISC_FEATURE (4)..(4): Xaa is Ser or Ala
QQRXNWPT

SEQ ID NO: 20 - protein, 8 aa, Artificial Sequence
  Synthetic
QQRSNWPT

SEQ ID NO: 21 - protein, 8 aa, Artificial Sequence
  Synthetic
QQRANWPT

SEQ ID NO: 22 - protein, 123 aa, Artificial Sequence
  Synthetic
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 23 - protein, 106 aa, Artificial Sequence
  Synthetic
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 24 - protein, 123 aa, Artificial Sequence; identical to SEQ ID NO: 22
  Synthetic

SEQ ID NO: 25 - protein, 106 aa, Artificial Sequence
  Synthetic
EIVLTQSPATLSLSPGERATLSCRASQSVSSYLAWYQQKPGQAPRLLIYDASNRAPGIPARFSGSGSGTDFTLTISSLEPEDFAVYYCQQRSNWPTFGQGTKVEIK

SEQ ID NO: 26 - protein, 123 aa, Artificial Sequence
  Synthetic
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVRGSPFGMDVWGQGTTVTVSS

SEQ ID NO: 27 - protein, 106 aa, Artificial Sequence; identical to SEQ ID NO: 23
  Synthetic

SEQ ID NO: 28 - protein, 123 aa, Artificial Sequence
  Synthetic
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFRFVSGSPFGMDVWGQGTTVTVSS

SEQ ID NO: 29 - protein, 106 aa, Artificial Sequence; identical to SEQ ID NO: 23
  Synthetic

SEQ ID NO: 30 - protein, 123 aa, Artificial Sequence
  Synthetic
QVQLVQSGAEVKKPGSSVKVSCKTSGDTFSTYAISWVRQAPGQGLEWMGGIIPLFGKAHYAQKFQGRVTITADESTSTAYMELSSLRSEDTAVYFCARKFHFVSGSPFGMDVWGQGTTVTVSS

SEQ ID NO: 31 - protein, 106 aa, Artificial Sequence; identical to SEQ ID NO: 23
  Synthetic

SEQ ID NO: 32 - protein, 123 aa, Artificial Sequence; identical to SEQ ID NO: 22
  Synthetic

SEQ ID NO: 33 - protein, 106 aa, Artificial Sequence
  Synthetic
EIVLTQSPATLSLSPGERATLSCRASQSVSSYLAWYQQKPGQAPRLLIYDASNRATGIPARFSGSGSGTDFTLTISSLEPEDFAVYYCQQRANWPTFGQGTKVEIK

SEQ ID NO: 34 - protein, 123 aa, Artificial Sequence; identical to SEQ ID NO: 22
  Synthetic

SEQ ID NO: 35 - protein, 106 aa, Artificial Sequence
  Synthetic
EIVLTQSPATLSLSPGERATLSCRASQSVSSYLAWYQQKPGQAPRLLIYDASNRAMGIPARFSGSGSGTDFTLTISSLEPEDFAVYYCQQRSNWPTFGQGTKVEIK

SEQ ID NO: 36 - protein, 123 aa, Artificial Sequence; identical to SEQ ID NO: 22
  Synthetic

SEQ ID NO: 37 - protein, 106 aa, Artificial Sequence
  Synthetic
EIVLTQSPATLSLSPGERATLSCRASQSVSSYLSWYQQKPGQAPRLLIYDASNRAPGIPARFSGSGSGTDFTLTISSLEPEDFAVYYCQQRSNWPTFGQGTKVEIK

SEQ ID NO: 38 - protein, 123 aa, Artificial Sequence; identical to SEQ ID NO: 22
  Synthetic

SEQ ID NO: 39 - protein, 106 aa, Artificial Sequence
  Synthetic
EIVLTQSPATLSLSPGERATLSCRASQSVSSYMAWYQQKPGQAPRLLIYDASNRAEGIPARFSGSGSGTDFTLTISSLEPEDFAVYYCQQRANWPTFGQGTKVEIK

SEQ ID NO: 40 - protein, 123 aa, Artificial Sequence; identical to SEQ ID NO: 22
  Synthetic

SEQ ID NO: 41 - protein, 106 aa, Artificial Sequence
  Synthetic
EIVLTQSPATLSLSPGERATLSCRASQSVSSYLEWYQQKPGQAPRLLIYDASNRATGIPARFSGSGSGTDFTLTISSLEPEDFAVYYCQQRSNWPTFGQGTKVEIK

SEQ ID NO: 42 - protein, 123 aa, Artificial Sequence; identical to SEQ ID NO: 30
  Synthetic

SEQ ID NO: 43 - protein, 106 aa, Artificial Sequence; identical to SEQ ID NO: 25
  Synthetic

SEQ ID NO: 44 - protein, 123 aa, Artificial Sequence; identical to SEQ ID NO: 26
  Synthetic

SEQ ID NO: 45 - protein, 106 aa, Artificial Sequence; identical to SEQ ID NO: 25
  Synthetic

SEQ ID NO: 46 - protein, 123 aa, Artificial Sequence
  Synthetic
QVQLVQSGAEVKKPGSSVKVSCKTSGDTFSTYAISWVRQAPGQGLEWMGGIIPLFGKAHYAQKFQGRVTITADESTSTAYMELSSLRSEDTAVYFCARKFHFVRGSPFGMDVWGQGTTVTVSS

SEQ ID NO: 47 - protein, 106 aa, Artificial Sequence; identical to SEQ ID NO: 23
  Synthetic

SEQ ID NO: 48 - protein, 123 aa, Artificial Sequence; identical to SEQ ID NO: 46
  Synthetic

SEQ ID NO: 49 - protein, 106 aa, Artificial Sequence; identical to SEQ ID NO: 25
  Synthetic

SEQ ID NO: 50 - protein, 106 aa, Artificial Sequence; identical to SEQ ID NO: 23
  Synthetic

SEQ ID NO: 51 - protein, 106 aa, Artificial Sequence; identical to SEQ ID NO: 25
  Synthetic

SEQ ID NO: 52 - protein, 106 aa, Artificial Sequence; identical to SEQ ID NO: 23
  Synthetic

SEQ ID NO: 53 - protein, 106 aa, Artificial Sequence; identical to SEQ ID NO: 23
  Synthetic

SEQ ID NO: 54 - protein, 106 aa, Artificial Sequence; identical to SEQ ID NO: 23
  Synthetic

SEQ ID NO: 55 - protein, 106 aa, Artificial Sequence; identical to SEQ ID NO: 33
  Synthetic

SEQ ID NO: 56 - protein, 106 aa, Artificial Sequence; identical to SEQ ID NO: 35
  Synthetic

SEQ ID NO: 57 - protein, 106 aa, Artificial Sequence; identical to SEQ ID NO: 37
  Synthetic

SEQ ID NO: 58 - protein, 106 aa, Artificial Sequence; identical to SEQ ID NO: 39
  Synthetic

SEQ ID NO: 59 - protein, 106 aa, Artificial Sequence; identical to SEQ ID NO: 41
  Synthetic

SEQ ID NO: 60 - protein, 106 aa, Artificial Sequence; identical to SEQ ID NO: 25
  Synthetic

SEQ ID NO: 61 - protein, 106 aa, Artificial Sequence; identical to SEQ ID NO: 25
  Synthetic

SEQ ID NO: 62 - protein, 106 aa, Artificial Sequence; identical to SEQ ID NO: 23
  Synthetic

SEQ ID NO: 63 - protein, 106 aa, Artificial Sequence; identical to SEQ ID NO: 25
  Synthetic

SEQ ID NO: 64 - protein, 123 aa, Artificial Sequence; identical to SEQ ID NO: 22
  Synthetic

SEQ ID NO: 65 - protein, 123 aa, Artificial Sequence; identical to SEQ ID NO: 22
  Synthetic

SEQ ID NO: 66 - protein, 123 aa, Artificial Sequence; identical to SEQ ID NO: 26
  Synthetic

SEQ ID NO: 67 - protein, 123 aa, Artificial Sequence; identical to SEQ ID NO: 28
  Synthetic

SEQ ID NO: 68 - protein, 123 aa, Artificial Sequence; identical to SEQ ID NO: 30
  Synthetic

SEQ ID NO: 69 - protein, 123 aa, Artificial Sequence; identical to SEQ ID NO: 22
  Synthetic

SEQ ID NO: 70 - protein, 123 aa, Artificial Sequence; identical to SEQ ID NO: 22
  Synthetic

SEQ ID NO: 71 - protein, 123 aa, Artificial Sequence; identical to SEQ ID NO: 22
  Synthetic

SEQ ID NO: 72 - protein, 123 aa, Artificial Sequence; identical to SEQ ID NO: 22
  Synthetic

SEQ ID NO: 73 - protein, 123 aa, Artificial Sequence; identical to SEQ ID NO: 22
  Synthetic

SEQ ID NO: 74 - protein, 123 aa, Artificial Sequence; identical to SEQ ID NO: 30
  Synthetic

SEQ ID NO: 75 - protein, 123 aa, Artificial Sequence; identical to SEQ ID NO: 26
  Synthetic

SEQ ID NO: 76 - protein, 123 aa, Artificial Sequence; identical to SEQ ID NO: 46
  Synthetic

SEQ ID NO: 77 - protein, 123 aa, Artificial Sequence; identical to SEQ ID NO: 46
  Synthetic

(19) United States
(12) Patent Application Publication
(10) Pub. No.: US 2024/0287189 A1
(43) Pub. Date: Aug. 29, 2024

(54) ANTI-PDL1 ANTIBODIES AND USES THEREOF

(71) Applicant: SPARX BIOSCIENCE LIMITED, Road Town, Tortola (VG)

(72) Inventors: Guidong Zhu, Gurnee, IL (US); Jingdong Ye, Vernon Hills, IL (US); Jingdong Qin, Woodridge, IL (US); Jichun Ma, Germantown, MD (US)

(73) Assignee: SPARX BIOSCIENCE LIMITED, Road Town, Tortola (VG)

(21) Appl. No.: 18/570,249

(22) PCT Filed: Jun. 17, 2022
(86) PCT No.: PCT/US22/33961

§ 371 Date: Dec. 14, 2023

Related U.S. Application Data
(63) Continuation of application No. 17/842,733, filed on Jun. 16, 2022, now abandoned.
(60) Provisional application No. 63/211,794, filed on Jun. 17, 2021.

(51) Int. Cl. C07K 16/28; A61K 39/00

(52) U.S. Cl. CPC C07K 16/2827; C07K 16/2818; A61K 2039/505; C07K 2317/21; C07K 2317/55; C07K 2317/622; C07K 2317/73; C07K 2317/76; C07K 2317/92

(57) ABSTRACT

Antibodies specifically binding to human program death-ligand 1 (PDL1) with high binding affinity, pharmaceutical compositions comprising such, and methods of using such for treating a target disease such as cancer.

### CROSS-REFERENCE TO RELATED APPLICATIONS

This application claims the benefit of priority to U.S. Provisional Application No. 63/211,794, filed on Jun. 17, 2021 and U.S. patent application Ser. No. 17/842,733, filed on Jun. 16, 2022, each of which is hereby incorporated by reference in its entirety.

### SEQUENCE LISTING

The instant application contains a Sequence Listing which has been filed electronically in ASCII format and is hereby incorporated by reference in its entirety. Said ASCII copy, created on Jun. 10, 2022, is named 112124-0020-70001WO00_SEQ.txt and is 65,515 bytes in size.

### BACKGROUND OF THE INVENTION

Programmed cell death protein 1 (PD-1) is a cell surface receptor expressed on immune cells and acts as an immune checkpoint that negatively regulates immune responses. Programmed cell death-ligand 1 (PDL1) is one of the ligand to PD-1. Binding of PDL1 to PD-1 triggers the PD-1 mediated signaling pathway, leading to negative regulation of immune responses.

Given the immune suppressive roles, PD1/PDL1 inhibitors are believed to activate immune responses against, for example, cancer cells. Indeed, antibodies against PD1 and PDL1 have shown clinical benefits in treating various types of cancer.

### SUMMARY OF THE INVENTION

The present disclosure is based on the development of anti-PDL1 antibodies having enhanced binding affinity to human PDL1. Such anti-PDL1 antibodies are expected to exhibit superior effects in activating immune responses and treating diseases such as cancer.

Accordingly, one aspect of the present disclosure features an isolated antibody that binds human programmed death-ligand 1 (PDL1). The anti-PDL1 antibody disclosed herein may comprise (a) a heavy chain variable region comprising (i) a heavy chain complementary determining region (CDR) 1 that comprises the amino acid sequence of GDTFSTYAIS (SEQ ID NO:1), (ii) a heavy chain CDR2 that comprises the amino acid sequence of GIIPX₁FGKAH (SEQ ID NO: 2), in which X1 is I or L, and (iii) a heavy chain CDR3 that comprises the amino acid sequence of KFX₂FVX₃GSPFGMDV (SEQ ID NO: 5), in which X₂ is Hor R and X₃ is S or R. Alternatively or in addition, the anti-PDL1 antibody may comprise (b) a light chain variable region comprising (i) a light chain CDR1 that comprises the amino acid sequence of RASQSVSSYX₄X₅ (SEQ ID NO: 9), in which X₄ is L or M and X₅ is A, S, or E, (ii) a light chain CDR2 that comprises the amino acid sequence of DASNRAX₆ (SEQ ID NO: 14), in which X₆ is T, P, M, or E, and (iii) a light chain CDR3 that comprises the amino acid sequence of QQRX-NWPT (SEQ ID NO: 19), in which X₇ is S or A. In some embodiments, the antibody is not 12A4.

In some embodiments, the heavy chain CDR2 is GIIPIFGKAHYAQKFQG (SEQ ID NO:3) or GIIPLFGKAHYAQKFQG (SEQ ID NO: 4). Alternatively or in addition, the heavy chain CDR3 is KFHFVSGSPFGMDV (SEQ ID NO:6), KFHFVRGSPFGMDV (SEQ ID NO:7), or KFRFVSGSPFGMDV (SEQ ID NO:8).

In some embodiments, the light chain CDR1 is RASQSVSSYLA (SEQ ID NO:10), RASQSVSSYLS (SEQ ID NO:11), RASQSVSSYMA (SEQ ID NO:12), or RASQSVSSYLE (SEQ ID NO:13). Alternatively or in addition, the light chain CDR2 is DASNRAT (SEQ ID NO:15), DASNRAP (SEQ ID NO:16), DASNRAM (SEQ ID NO:17), or DASNRAE (SEQ ID NO:18). Alternatively or in addition, the light chain CDR3 is QQRSNWPT (SEQ ID NO:20) or QQRANWPT (SEQ ID NO:21).

In some embodiments, the anti-PDL1 antibody disclosed herein may comprise the same heavy chain CDR1-CDR3 and/or the same light chain CDR1-CDR3 as antibody 12A4a, 12A4b, 12A4c, 12A4d, 12A4e, 12A4f, 12A4g, 12A4h, 12A4i, 12A4ad, 12A4ba, 12A4bd, or 12A4bad. In some examples, the anti-PDL1 antibody may comprise the same heavy chain variable region and/or the same light chain variable region as antibody 12A4a, 12A4b, 12A4c, 12A4d, 12A4e, 12A4f, 12A4g, 12A4h, 12A4i, 12A4ad, 12A4ba, 12A4bd, or 12A4bad.

Any of the anti-PDL1 antibodies disclosed herein can be a full-length antibody or an antigen binding fragment thereof. Alternatively, the antibody can be a single-chain variable fragment (scFv). Any of the anti-PDL1 antibodies disclosed herein may be a human antibody or a humanized antibody.

In another aspect, provided herein is a nucleic acid or a set of nucleic acids, which collectively encode any of the anti-PDL1 antibodies disclosed herein. In some embodiments, the nucleic acid or set of nucleic acids can be a vector or a set of vectors, for example, expression vector(s).

Also provided herein are host cells comprising the nucleic acid or the set of nucleic acids coding for the anti-PDL1 antibodies disclosed herein. Such host cells can be bacterial cells, yeast cells, insect cells, or mammalian cells.

Further, the present disclosure provides a pharmaceutical composition comprising any of the anti-PDL1 antibodies, or the coding nucleic acid(s) thereof and a pharmaceutically acceptable carrier. The present disclosure also provides a method for inhibiting PDL1-positive cells, comprising administering to a subject in need thereof an effective amount of the pharmaceutical composition. In some instances, the subject is a human patient having cancer.

In addition, the present disclosure provides a method for producing an antibody binding to human PDL1, the method comprising: (i) culturing a host cell comprising coding nucleic acid(s) of the anti-PDL1 antibody as disclosed herein under conditions allowing for expression of the antibody; and (ii) harvesting the antibody thus produced.

Also within the scope of the present disclosure are pharmaceutical compositions comprising any of the anti-PDL1 antibodies or coding nucleic acid(s) thereof for use in treating a disorder associated with the PD1-PDL1 signaling such as cancer and uses of the antibody or the coding nucleic acid(s) for manufacturing a medicament for treating the target diseases.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIG. 1 is a diagram showing binding of exemplary anti-PDL1 antibody 12A4bad to human PDL1 relative to parent clone 12A4 as determined by competitive phage ELISA.

FIG. 2 is a graph showing cell-based affinity determination of exemplary anti-PDL1 monoclonal antibody 12A4bad relative to durvalumab. The mAbs tested exhibited dose-dependent specific binding to human breast cancer cell line MDA MB-231 which highly express PDL1.

FIG. 3 is a graph showing results of PDL1/PD1 blockage bioassay of exemplary anti-PDL1 monoclonal antibody 12A4bad relative to durvalumab. The monoclonal antibody 12A4bad could effectively block PDL1-PD1 binding in two genetically engineered cell lines, PD-1 effector cells and PDL1 aAPC/CHO-K1 cells.

### DETAILED DESCRIPTION OF THE INVENTION

Provided herein are antibodies having high binding affinity to human PDL1 (“anti-PDL1 antibodies”). The anti-PDL1 antibodies disclosed herein show high binding affinity to PDL1 as relative to the parent 12A4 anti-PDL1 antibody.

Programmed death-ligand 1 (PDL1), also known as CD274 or B7 homolog 1, is an about 40 kDa type 1 transmembrane protein that plays a role in suppressing the adaptive arm of the immune system under certain circumstances. PDL1 molecules from various species are well known in the art. PDL1 binds to its receptor, PD-1, found on activated T cells, B cells, and myeloid cells, to modulate activation or inhibition. It was reported that upregulation of PDL1 may allow cancer cells to evade the host immune systems. Anti-PDL1 antibodies have been reported as promising anti-cancer agents.

Thus, the anti-PDL1 antibodies disclosed herein can serve as therapeutic agents for treating diseases associated with the PD1-PDL1 signaling, for example, various types of cancer. The antibodies disclosed herein may also be used for research purposes.

### I. High Affinity Anti-PDL1 Antibodies

The present disclosure provides antibodies binding to PDL1, for example, human PDL1. In some embodiments, the anti-PDL1 antibodies disclosed herein are capable of binding to PDL1 expressed on cell surface. As such, the antibodies disclosed herein may be used for either therapeutic or diagnostic purposes to block PD1-PDL1 interaction and/or target immune cells expressing PDL1. As used herein, the term “anti-PDL1 antibody” refers to any antibody capable of binding to a PDL1 polypeptide, which can be of a suitable source, for example, human or a non-human mammal (e.g., mouse, rat, rabbit, primate such as monkey, etc.).

An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody”, e.g., anti-PDL1 antibody, encompasses not only intact (e.g., full-length) polyclonal or monoclonal antibodies, but also antigen-binding fragments thereof (such as Fab, Fab′, F(ab′)2, Fv), single-chain antibody (scFv), fusion proteins comprising an antibody portion, humanized antibodies, chimeric antibodies, diabodies, single domain antibody (e.g., nanobody), single domain antibodies (e.g., a V_{H} only antibody), multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody, e.g., anti-Galectin-9 antibody, includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

A typical antibody molecule comprises a heavy chain variable region (V_{H}) and a light chain variable region (V_{L}), which are usually involved in antigen binding. The V_{H} and V_{L} regions can be further subdivided into regions of hypervariability, also known as “complementarity determining regions” (“CDR”), interspersed with regions that are more conserved, which are known as “framework regions” (“FR”). Each V_{H} and V_{L} is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinf.org.uk/abs).

The anti-PDL1 antibody described herein may be a full-length antibody, which contains two heavy chains and two light chains, each including a variable domain and a constant domain. Alternatively, the anti-PDL1 antibody can be an antigen-binding fragment of a full-length antibody. Examples of binding fragments encompassed within the term “antigen-binding fragment” of a full length antibody include (i) a Fab fragment, a monovalent fragment consisting of the V_{L}, V_{H}, CL and CH1 domains; (ii) a F(ab′)2 fragment, a bivalent fragment including two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR) that retains functionality. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv). See e.g., Bird et al. (1988) Science 242:423-426; and Huston et al. (1988) Proc. Natl. Acad. Sci. USA 85:5879-5883.

The antibodies described herein can be of a suitable origin, for example, murine, rat, or human. Such antibodies are non-naturally occurring, i.e., would not be produced in an animal without human act (e.g., immunizing such an animal with a desired antigen or fragment thereof or isolated from antibody libraries). Any of the antibodies described herein, e.g., anti-PDL1 antibody, can be either monoclonal or polyclonal. A “monoclonal antibody” refers to a homogenous antibody population and a “polyclonal antibody” refers to a heterogeneous antibody population. These two terms do not limit the source of an antibody or the manner in which it is made.

In some embodiments, the anti-PDL1 antibodies may be humanized antibodies or chimeric antibodies. Humanized antibodies refer to forms of non-human (e.g., murine) antibodies that are specific chimeric immunoglobulins, immunoglobulin chains, or antigen-binding fragments thereof that contain minimal sequence derived from non-human immunoglobulin. In general, humanized antibodies are human immunoglobulins (recipient antibody) in which residues from a CDR of the recipient are replaced by residues from a CDR of a non-human species (donor antibody) such as mouse, rat, or rabbit having the desired specificity, affinity, and capacity. In some instances, one or more Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In some instances, the humanized antibody may comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more CDRs (one, two, three, four, five, or six) which are altered with respect to the original antibody, which are also termed one or more CDRs “derived from” one or more CDRs from the original antibody. Humanized antibodies may also involve affinity maturation. Methods for constructing humanized antibodies are also well known in the art. See, e.g., Queen et al., Proc. Natl. Acad. Sci. USA, 86:10029-10033 (1989).

In some embodiments, the anti-PDL1 antibody disclosed herein can be a chimeric antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region. Techniques developed for the production of “chimeric antibodies” are well known in the art. See, e.g., Morrison et al. (1984) Proc. Natl. Acad. Sci. USA 81, 6851; Neuberger et al. (1984) Nature 312, 604; and Takeda et al. (1984) Nature 314:452.

In some embodiments, an anti-PDL1 antibody as described herein has a suitable binding affinity for the target antigen (e.g., PDL1) or antigenic epitopes thereof. As used herein, “binding affinity” refers to the apparent association constant or K_{A}. The K_{A} is the reciprocal of the dissociation constant (K_{D}). The anti-PDL1 antibody described herein may have a binding affinity (K_{D}) of at least 100 nM, 10 nM, InM, 0.1 nM, or lower for PDL1. An increased binding affinity corresponds to a decreased K_{D}. Higher affinity binding of an antibody for a first antigen relative to a second antigen can be indicated by a higher K_{A} (or a smaller numerical value K_{D}) for binding the first antigen than the K_{A} (or numerical value K_{D}) for binding the second antigen. In such cases, the antibody has specificity for the first antigen (e.g., a first protein in a first conformation or mimic thereof) relative to the second antigen (e.g., the same first protein in a second conformation or mimic thereof; or a second protein). Differences in binding affinity (e.g., for specificity or other comparisons) can be at least 1.5, 2, 3, 4, 5, 10, 15, 20, 37.5, 50, 70, 80, 90, 100, 500, 1000, 10,000 or 10⁵ fold. In some embodiments, any of the anti-PDL1 antibodies may be further affinity matured to increase the binding affinity of the antibody to the target antigen or antigenic epitope thereof.

Binding affinity (or binding specificity) can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in HBS-P buffer (10 mM HEPES pH7.4, 150 mM NaCl, 0.005% (v/v) Surfactant P20). These techniques can be used to measure the concentration of bound binding protein as a function of target protein concentration. The concentration of bound binding protein ([Bound]) is generally related to the concentration of free target protein ([Free]) by the following equation:

$$[ Bound ] = [ Free ] / ( Kd + [ Free ] )$$

It is not always necessary to make an exact determination of K_{A}, though, since sometimes it is sufficient to obtain a quantitative measurement of affinity, e.g., determined using a method such as ELISA or FACS analysis, is proportional to K_{A}, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

In some embodiments, the anti-PDL1 antibody disclosed herein has an EC₅₀ value of lower than 10 nM, e.g., <5 nM, <2 nM, or <InM for binding to PDL1-positive cells. As used herein, EC₅₀ values refer to the minimum concentration of an antibody required to bind to 50% of the cells in a PDL1-positive cell population. EC₅₀ values can be determined using conventional assays and/or assays disclosed herein. See, e.g., Example 1 below.

A number of exemplary anti-PDL1 antibodies are provided in Table 1 below (CDRs identified as determined by the Kabat approach. See, e.g., Kabat, E. A., et al. (1991) Sequences of Proteins of Immunological Interest, Fifth Edition, U.S. Department of Health and Human Services, NIH Publication No. 91-3242. See also www2.mrc-lmb.cam.ac.uk/vbase/alignments2.php. 2018EP187-A12 (a.k.a., EP187-A12)

TABLE 1
Exemplary Anti-PDLl Antibodies

| | | | SEQ |
|---|---|---|---|
| | | Sequence | ID NO |
| VH CDR1 | | GDTFSTYAIS | 1 |
| VH CDR2 | | GIIPX₁FGKAH (X₁ is I or L) | 2 |
| | | GIIPIFGKAHYAQKFQG | 3 |
| | | GIIPLFGKAHYAQKFQG | 4 |
| VH CDR3 | | KFX₂FVX₃GSPFGMDV (X₂ is H or R; X₃ is S or R) | 5 |
| | | KFHFVSGSPFGMDV | 6 |
| | | KFHFVRGSPFGMDV | 7 |
| | | KFRFVSGSPFGMDV | 8 |
| VL CDR1 | | RASQSVSSYX₄X₅ (X₄ is L or M; X₅ is A, S, or E) | 9 |
| | | RASQSVSSYLA | 10 |
| | | RASQSVSSYLS | 11 |
| | | RASQSVSSYMA | 12 |
| | | RASQSVSSYLE | 13 |
| VL CDR2 | | DASNRAX₆ (X₆ is T, P, M, or E) | 14 |
| | | DASNRAT | 15 |
| | | DASNRAP | 16 |
| | | DASNRAM | 17 |
| | | DASNRAE | 18 |
| VL CDR3 | | QQRX₇NWPT (X₇ is S or A) | 19 |
| | | QQRSNWPT | 20 |
| | | QQRANWPT | 21 |
| 12A4 | VH | QVQLVQSGAEVKKPGSSVKVSCKTSGDTFSTYAISWVRQAPGQGLEWMGG | 22 |
| | | IIPIFGKAHYAQKFQGRVTITADESTSTAYMELSSLRSEDTAVYFCARKF | |
| | | HFVSGSPFGMDVWGQGTTVTVSS | |
| | VL | EIVLTQSPATLSLSPGERATLSCRASQSVSSYLAWYQQKPGQAPRLLIYD | 23 |
| | | ASNRATGIPARFSGSGSGTDFTLTISSLEPEDFAVYYCQQRSNWPTFGQG | |
| | | TKVEIK | |
| 12A4a | VH | QVQLVQSGAEVKKPGSSVKVSCKTSGDTFSTYAISWVRQAPGQGLEWMGG | 24 |
| | | IIPIFGKAHYAQKFQGRVTITADESTSTAYMELSSLRSEDTAVYFCARKF | |
| | | HFVSGSPFGMDVWGQGTTVTVSS | |
| | VL | EIVLTQSPATLSLSPGERATLSCRASQSVSSYLAWYQQKPGQAPRLLIYD | 25 |
| | | ASNRAPGIPARFSGSGSGTDFTLTISSLEPEDFAVYYCQQRSNWPTFGQG | |
| | | TKVEIK | |
| 12A4b | VH | QVQLVQSGAEVKKPGSSVKVSCKTSGDTFSTYAISWVRQAPGQGLEWMGG | 26 |
| | | IIPIFGKAHYAQKFQGRVTITADESTSTAYMELSSLRSEDTAVYFCARKF | |
| | | HFVRGSPFGMDVWGQGTTVTVSS | |
| | VL | EIVLTQSPATLSLSPGERATLSCRASQSVSSYLAWYQQKPGQAPRLLIYD | 27 |
| | | ASNRATGIPARFSGSGSGTDFTLTISSLEPEDFAVYYCQQRSNWPTFGQG | |
| | | TKVEIK | |
| 12A4c | VH | QVQLVQSGAEVKKPGSSVKVSCKTSGDTFSTYAISWVRQAPGQGLEWMGG | 28 |
| | | IIPIFGKAHYAQKFQGRVTITADESTSTAYMELSSLRSEDTAVYFCARKF | |
| | | HFVSGSPFGMDVWGQGTTVTVSS | |
| | VL | EIVLTQSPATLSLSPGERATLSCRASQSVSSYLAWYQQKPGQAPRLLIYD | 29 |
| | | ASNRATGIPARFSGSGSGTDFTLTISSLEPEDFAVYYCQQRSNWPTFGQG | |
| | | TKVEIK | |
| 12A4d | VH | QVQLVQSGAEVKKPGSSVKVSCKTSGDTFSTYAISWVRQAPGQGLEWMGG | 30 |
| | | IIPLFGKAHYAQKFQGRVTITADESTSTAYMELSSLRSEDTAVYFCARKF | |
| | | HFVSGSPFGMDVWGQGTTVTVSS | |
| | VL | EIVLTQSPATLSLSPGERATLSCRASQSVSSYLAWYQQKPGQAPRLLIYD | 31 |
| | | ASNRATGIPARFSGSGSGTDFTLTISSLEPEDFAVYYCQQRSNWPTFGQG | |
| | | TKVEIK | |
| 12A4e | VH | QVQLVQSGAEVKKPGSSVKVSCKTSGDTFSTYAISWVRQAPGQGLEWMGG | 32 |
| | | IIPIFGKAHYAQKFQGRVTITADESTSTAYMELSSLRSEDTAVYFCARKF | |
| | | HFVSGSPFGMDVWGQGTTVTVSS | |
| | VL | EIVLTQSPATLSLSPGERATLSCRASQSVSSYLAWYQQKPGQAPRLLIYD | 33 |
| | | ASNRATGIPARFSGSGSGTDFTLTISSLEPEDFAVYYCQQRANWPTFGQG | |
| | | TKVEIK | |
| 12A4f | VH | QVQLVQSGAEVKKPGSSVKVSCKTSGDTFSTYAISWVRQAPGQGLEWMGG | 34 |
| | | IIPIFGKAHYAQKFQGRVTITADESTSTAYMELSSLRSEDTAVYFCARKF | |
| | | HFVSGSPFGMDVWGQGTTVTVSS | |
| | VL | EIVLTQSPATLSLSPGERATLSCRASQSVSSYLAWYQQKPGQAPRLLIYD | 35 |
| | | ASNRAMGIPARFSGSGSGTDFTLTISSLEPEDFAVYYCQQRSNWPTFGQG | |
| | | TKVEIK | |
| 12A4g | VH | QVQLVQSGAEVKKPGSSVKVSCKTSGDTFSTYAISWVRQAPGQGLEWMGG | 36 |
| | | IIPIFGKAHYAQKFQGRVTITADESTSTAYMELSSLRSEDTAVYFCARKF | |
| | | HFVSGSPFGMDVWGQGTTVTVSS | |
| | VL | EIVLTQSPATLSLSPGERATLSCRASQSVSSYLSWYQQKPGQAPRLLIYD | 37 |
| | | ASNRAPGIPARFSGSGSGTDFTLTISSLEPEDFAVYYCQQRSNWPTFGQG | |
| | | TKVEIK | |
| 12A4h | VH | QVQLVQSGAEVKKPGSSVKVSCKTSGDTFSTYAISWVRQAPGQGLEWMGG | 38 |
| | | IIPIFGKAHYAQKFQGRVTITADESTSTAYMELSSLRSEDTAVYFCARKF | |
| | | HFVSGSPFGMDVWGQGTTVTVSS | |
| | VL | EIVLTQSPATLSLSPGERATLSCRASQSVSSYMAWYQQKPGQAPRLLIYD | 39 |
| | | ASNRAEGIPARFSGSGSGTDFTLTISSLEPEDFAVYYCQQRANWPTFGQG | |
| | | TKVEIK | |
| 12A4i | VH | QVQLVQSGAEVKKPGSSVKVSCKTSGDTFSTYAISWVRQAPGQGLEWMGG | 40 |
| | | IIPIFGKAHYAQKFQGRVTITADESTSTAYMELSSLRSEDTAVYFCARKF | |
| | | HFVSGSPFGMDVWGQGTTVTVSS | |
| | VL | EIVLTQSPATLSLSPGERATLSCRASQSVSSYLEWYQQKPGQAPRLLIYD | 41 |
| | | ASNRATGIPARFSGSGSGTDFTLTISSLEPEDFAVYYCQQRSNWPTFGQG | |
| | | TKVEIK | |
| 12A4ad | VH | QVQLVQSGAEVKKPGSSVKVSCKTSGDTFSTYAISWVRQAPGQGLEWMGG | 42 |
| | | IIPLFGKAHYAQKFQGRVTITADESTSTAYMELSSLRSEDTAVYFCARKF | |
| | | HFVSGSPFGMDVWGQGTTVTVSS | |
| | VL | EIVLTQSPATLSLSPGERATLSCRASQSVSSYLAWYQQKPGQAPRLLIYD | 43 |
| | | ASNRAPGIPARFSGSGSGTDFTLTISSLEPEDFAVYYCQQRSNWPTFGQG | |
| | | TKVEIK | |
| 12A4ba | VH | QVQLVQSGAEVKKPGSSVKVSCKTSGDTFSTYAISWVRQAPGQGLEWMGG | 44 |
| | | IIPIFGKAHYAQKFQGRVTITADESTSTAYMELSSLRSEDTAVYFCARKF | |
| | | HFVRGSPFGMDVWGQGTTVTVSS | |
| | VL | EIVLTQSPATLSLSPGERATLSCRASQSVSSYLAWYQQKPGQAPRLLIYD | 45 |
| | | ASNRAPGIPARFSGSGSGTDFTLTISSLEPEDFAVYYCQQRSNWPTFGQG | |
| | | TKVEIK | |
| 12A4bd | VH | QVQLVQSGAEVKKPGSSVKVSCKTSGDTFSTYAISWVRQAPGQGLEWMGG | 46 |
| | | IIPLFGKAHYAQKFQGRVTITADESTSTAYMELSSLRSEDTAVYFCARKF | |
| | | HFVRGSPFGMDVWGQGTTVTVSS | |
| | VL | EIVLTQSPATLSLSPGERATLSCRASQSVSSYLAWYQQKPGQAPRLLIYD | 47 |
| | | ASNRATGIPARFSGSGSGTDFTLTISSLEPEDFAVYYCQQRSNWPTFGQG | |
| | | TKVEIK | |
| 12A4bad | VH | QVQLVQSGAEVKKPGSSVKVSCKTSGDTFSTYAISWVRQAPGQGLEWMGG | 48 |
| | | IIPLFGKAHYAQKFQGRVTITADESTSTAYMELSSLRSEDTAVYFCARKF | |
| | | HFVRGSPFGMDVWGQGTTVTVSS | |
| | VL | EIVLTQSPATLSLSPGERATLSCRASQSVSSYLAWYQQKPGQAPRLLIYD | 49 |
| | | ASNRAPGIPARFSGSGSGTDFTLTISSLEPEDFAVYYCQQRSNWPTFGQG | |
| | | TKVEIK | |

In some embodiments, The anti-PDL1 antibody disclosed herein may comprise (i) a heavy chain complementary determining region (CDR) 1 that comprises the amino acid sequence of GDTFSTYAIS (SEQ ID NO:1), (ii) a heavy chain CDR2 that comprises the amino acid sequence of GIIPX₁FGKAH (SEQ ID NO: 2), in which X₁ is I or L, and (iii) a heavy chain CDR3 that comprises the amino acid sequence of KFX₂FVX₃GSPFGMDV (SEQ ID NO: 5), in which X₂ is H or R and X₃ is S or R; and/or (iv) a light chain CDR1 that comprises the amino acid sequence of RASQSVSSYX₄X₅ (SEQ ID NO: 9), in which X₄ is L or M and X₅ is A, S, or E, (v) a light chain CDR2 that comprises the amino acid sequence of DASNRAX₆ (SEQ ID NO: 14), in which X₆ is T, P, M, or E, and (vi) a light chain CDR3 that comprises the amino acid sequence of QQRX-NWPT (SEQ ID NO: 19), in which X₇ is S or A.

In some examples, the heavy chain CDR2 in the anti-PDL1 antibody disclosed herein comprises the amino acid sequence of GIIPIFGKAHYAQKFQG (SEQ ID NO:3). In other examples, the heavy chain CDR2 can comprise the amino acid sequence of GIIPLFGKAHYAQKFQG (SEQ ID NO: 4).

In some examples, the heavy chain CDR3 of the anti-PDL1 antibody disclosed herein may comprise the amino acid sequence of KFHFVSGSPFGMDV (SEQ ID NO:6). In other examples, the heavy chain CDR3 may comprise the amino acid sequence of KFHFVRGSPFGMDV (SEQ ID NO:7). In yet other examples, the heavy chain CDR3 may comprise the amino acid sequence of KFRFVSGSPFGMDV (SEQ ID NO:8).

In some examples, the light chain CDR1 of the anti-PDL1 antibody may comprise the amino acid sequence of RASQSVSSYLA (SEQ ID NO:10). In other examples, the light chain CDR1 may comprise the amino acid sequence of RASQSVSSYLS (SEQ ID NO:11). Alternatively, the light chain CDR1 may comprise the amino acid sequence of RASQSVSSYMA (SEQ ID NO:12). In other examples, the light chain CDR1 may comprise the amino acid sequence of RASQSVSSYLE (SEQ ID NO:13).

In some examples, the light chain CDR2 of the anti-PDL1 antibody disclosed herein may comprise the amino acid sequence of DASNRAT (SEQ ID NO:15). In other examples, the light chain CDR2 may comprise the amino acid sequence of DASNRAP (SEQ ID NO:16). Alternatively, the light chain CDR2 may comprise the amino acid sequence of DASNRAM (SEQ ID NO:17). In yet other examples, the light chain CDR2 may comprise the amino acid sequence of DASNRAE (SEQ ID NO:18).

In some examples, the light chain CDR3 of the anti-PDL1 antibody disclosed herein may comprise the amino acid sequence of QQRSNWPT (SEQ ID NO:20). Alternatively, the light chain CDR3 may comprise the amino acid sequence of QQRANWPT (SEQ ID NO:21).

In some instances, the anti-PDL1 antibody disclosed herein may have a combination of any of the heavy chain CDR1-CDR3 regions disclosed herein and/or a combination of any of the light chain CDR1-CDR3 regions disclosed herein, provided that the antibody does not have the same heavy chain and light chain CDRs as antibody 12A4. In some examples, the anti-PDL1 antibody has the same heavy chain and/or light chain CDRs as the exemplary anti-PDL1 antibodies listed in Table 1 above, e.g., 12A4a, 12A4b, 12A4d, 12A4ad, 12A4ba, 12A4bd, or 12A4bad. In specific examples, the anti-PDL1 antibody has the same heavy chain and light chain CDRs as those of antibody 12A4bad.

In some instances, the anti-PDL1 antibody disclosed herein may have a combination of any V_{H} and V_{L} sequences provided in Table 1 above. In some examples, the anti-PDL1 antibody may comprise the same V_{H} and V_{L} sequences as the exemplary antibodies listed in Table 1 above, e.g., 12A4a, 12A4b, 12A4d, 12A4ad, 12A4ba, 12A4bd, or 12A4bad. In specific examples, the anti-PDL1 antibody has the same V_{H} and V_{L} as those of antibody 12A4bad.

Two antibodies having the same V_{H} and/or V_{L} CDRs means that their CDRs are identical when determined by the same approach (e.g., the Kabat approach, the Chothia approach, the AbM approach, the Contact approach, or the IMGT approach as known in the art. See, e.g., bioinf.org.uk/abs/). Such anti-PDL1 antibodies may have the same V_{H}, the same V_{L}, or both as compared to an exemplary antibody described herein.

Also within the scope of the present disclosure are functional variants of any of the exemplary anti-PDL1 antibodies as disclosed herein. Such functional variants are substantially similar to the exemplary antibody, both structurally and functionally. A functional variant comprises substantially the same V_{H} and V_{L} CDRs as the exemplary antibody. For example, it may comprise only up to 8 (e.g., 8, 7, 6, 5, 4, 3, 2, or 1) amino acid residue variations in the total CDR regions of the antibody and binds the same epitope of PDL1 with substantially similar affinity (e.g., having a K_{D} value in the same order). In some instances, the functional variants may have the same heavy chain CDR3 as the exemplary antibody, and optionally the same light chain CDR3 as the exemplary antibody. Alternatively or in addition, the functional variants may have the same heavy chain CDR2 as the exemplary antibody. Such an anti-PDL1 antibody may comprise a V_{H} fragment having CDR amino acid residue variations in only the heavy chain CDR1 as compared with the V_{H} of the exemplary antibody. In some examples, the anti-PDL1 antibody may further comprise a V_{L} fragment having the same V_{L} CDR3, and optionally same V_{L} CDR1 or V_{L} CDR2 as the exemplary antibody.

Alternatively or in addition, the amino acid residue variations can be conservative amino acid residue substitutions. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

In some embodiments, the anti-PDL1 antibody may comprise heavy chain CDRs that are at least 80% (e.g., 85%, 90%, 95%, or 98%) sequence identity, individually or collectively, as compared with the V_{H} CDRs of an exemplary antibody described herein. Alternatively or in addition, the anti-PDL1 antibody may comprise light chain CDRs that are at least 80% (e.g., 85%, 90%, 95%, or 98%) sequence identity, individually or collectively, as compared with the V_{L} CDRs as an exemplary antibody described herein. As used herein, “individually” means that one CDR of an antibody shares the indicated sequence identity relative to the corresponding CDR of the exemplary antibody. “Collectively” means that three V_{H} Or V_{L} CDRs of an antiody in combination share the indicated sequence identity relative the corresponding three V_{H} or V_{L} CDRs of the exemplary antibody in combination.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In some embodiments, the heavy chain of any of the anti-PDL1 antibodies as described herein may further comprise a heavy chain constant region (CH) or a portion thereof (e.g., CH1, CH2, CH3, or a combination thereof). The heavy chain constant region can of any suitable origin, e.g., human, mouse, rat, or rabbit. Alternatively or in addition, the light chain of the anti-PDL1 antibody may further comprise a light chain constant region (CL), which can be any CL known in the art. In some examples, the CL is a kappa light chain. In other examples, the CL is a lambda light chain. Antibody heavy and light chain constant regions are well known in the art, e.g., those provided in the IMGT database (www.imgt.org) or at www.vbase2.org/vbstat.php., both of which are incorporated by reference herein.

In some embodiments, the anti-PDL1 antibody disclosed herein may be a single chain antibody (scFv). A scFv antibody may comprise a V_{H} fragment and a V_{L} fragment, which may be linked via a flexible peptide linker. In some instances, the scFv antibody may be in the V_{H}→V_{L} orientation (from N-terminus to C-terminus). In other instances, the scFv antibody may be in the V_{L}→V_{H} orientation (from N-terminus to C-terminus). Exemplary scFv anti-PDL1 antibodies are provided below (CDRs in boldface and peptide linker in boldface and underlined).

In some embodiments, any of the anti-PD-L1 antibodies disclosed herein (e.g., 12A4bad) may be used to construct a bi-specific antibody or a multi-specific antibody having the anti-PD-L1 antibody (e.g., a V_{L} or V_{H} fragment, an scFv fragment, or a Fab) as a binding moiety. Accordingly, bi-specific or multi-specific antibodies comprising any of the anti-PD-L1 antibodies disclosed herein such as 12A4bad are also within the scope of the present disclosure.

Any of the anti-PDL1 antibody as described herein, e.g., the exemplary anti-PDL1 antibodies provided here, can bind and inhibit (e.g., reduce or eliminate) the activity of PDL1. In some embodiments, the anti-PDL1 antibody as described herein can bind and inhibit the activity of PDL1 by at least 30% (e.g., 35%, 40%, 45%, 50%, 60%, 70%, 80%, 90%, 95% or greater, including any increment therein). The inhibitory activity of an anti-PDL1 antibody described herein can be determined by routine methods known in the art, e.g., by an assay for measuring the K_{i,}^{app} value.

In some examples, the K_{i,}^{app} value of an antibody may be determined by measuring the inhibitory effect of different concentrations of the antibody on the extent of a relevant reaction; fitting the change in pseudo-first order rate constant (v) as a function of inhibitor concentration to the modified Morrison equation (Equation 1) yields an estimate of the apparent Ki value. For a competitive inhibitor, the K_{i,}^{app} can be obtained from the y-intercept extracted from a linear regression analysis of a plot of K_{i,}^{app} versus substrate concentration.

$$v = A \cdot \frac{( [ E ] - [ I ] - {K}_{i}^{app} ) + \sqrt{{( [ E ] - [ I ] - {K}_{i}^{app} )}^{2} + 4 [ E ] \cdot } {K}_{i}^{app}}{2} \qquad ( Equation 1 )$$

Where A is equivalent to vₒ/E, the initial velocity (vₒ) of the enzymatic reaction in the absence of inhibitor (I) divided by the total enzyme concentration (E). In some embodiments, the anti-PDL1 antibody described herein may have a K_{i,}^{app} value of 1000, 500, 100, 50, 40, 30, 20, 10, 5 pM or less for the target antigen or antigen epitope.

### II. Preparation of Anti-PDL1 Antibodies

Antibodies capable of binding PDL1 as described herein can be made by any method known in the art. See, for example, Harlow and Lane, (1998) Antibodies: A Laboratory Manual, Cold Spring Harbor Laboratory, New York. In some embodiments, the antibody may be produced by the conventional hybridoma technology. Alternatively, the anti-PDL1 antibody may be identified from a suitable library (e.g., a human antibody library).

In some examples, antibodies capable of binding to the target antigens as described herein (a PDL1 molecule) may be isolated from a suitable antibody library via routine practice or guidance provided herein (see Example 1 below). Antibody libraries can be used to identify proteins that bind to a target antigen (e.g., human PDL1 such as cell surface PDL1) via routine screening processes. In the selection process, the polypeptide component is probed with the target antigen or a fragment thereof and, if the polypeptide component binds to the target, the antibody library member is identified, typically by retention on a support. Retained display library members are recovered from the support and analyzed. The analysis can include amplification and a subsequent selection under similar or dissimilar conditions. For example, positive and negative selections can be alternated. The analysis can also include determining the amino acid sequence of the polypeptide component and purification of the polypeptide component for detailed characterization.

There are a number of routine methods known in the art to identify and isolate antibodies capable of binding to the target antigens described herein, including phage display, yeast display, ribosomal display, or mammalian display technology.

Antigen-binding fragments of an intact antibody (full-length antibody) can be prepared via routine methods. For example, F(ab′)2 fragments can be produced by pepsin digestion of an antibody molecule, and Fab fragments that can be generated by reducing the disulfide bridges of F(ab′)2 fragments.

Genetically engineered antibodies, such as humanized antibodies, chimeric antibodies, single-chain antibodies, and bi-specific antibodies, can be produced via, e.g., conventional recombinant technology. In one example, DNA encoding a monoclonal antibody specific to a target antigen can be readily isolated and sequenced using conventional procedures (e.g., by using oligonucleotide probes that are capable of binding specifically to genes encoding the heavy and light chains of the monoclonal antibodies). Once isolated, the DNA may be placed into one or more expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. See, e.g., PCT Publication No. WO 87/04462. The DNA can then be modified, for example, by substituting the coding sequence for human heavy and light chain constant domains in place of the homologous murine sequences, Morrison et al., (1984) Proc. Nat. Acad. Sci. 81:6851, or by covalently joining to the immunoglobulin coding sequence all or part of the coding sequence for a non-immunoglobulin polypeptide. In that manner, genetically engineered antibodies, such as “chimeric” or “hybrid” antibodies; can be prepared that have the binding specificity of a target antigen.

Techniques developed for the production of “chimeric antibodies” are well known in the art. See, e.g., Morrison et al. (1984) Proc. Natl. Acad. Sci. USA 81, 6851; Neuberger et al. (1984) Nature 312, 604; and Takeda et al. (1984) Nature 314:452.

Methods for constructing humanized antibodies are also well known in the art. See, e.g., Queen et al., Proc. Natl. Acad. Sci. USA, 86:10029-10033 (1989). In one example, variable regions of V_{H} and V_{L} of a parent non-human antibody are subjected to three-dimensional molecular modeling analysis following methods known in the art. Next, framework amino acid residues predicted to be important for the formation of the correct CDR structures are identified using the same molecular modeling analysis. In parallel, human V_{H} and V_{L} chains having amino acid sequences that are homologous to those of the parent non-human antibody are identified from any antibody gene database using the parent V_{H} and V_{L} sequences as search queries. Human V_{H} and V_{L} acceptor genes are then selected.

The CDR regions within the selected human acceptor genes can be replaced with the CDR regions from the parent non-human antibody or functional variants thereof. When necessary, residues within the framework regions of the parent chain that are predicted to be important in interacting with the CDR regions (see above description) can be used to substitute for the corresponding residues in the human acceptor genes.

A single-chain antibody can be prepared via recombinant technology by linking a nucleotide sequence coding for a heavy chain variable region and a nucleotide sequence coding for a light chain variable region. Preferably, a flexible linker is incorporated between the two variable regions. Alternatively, techniques described for the production of single chain antibodies (U.S. Pat. Nos. 4,946,778 and 4,704,692) can be adapted to produce a phage-display, yeast-display, mammalian cell-display, or mRNA-display scFv library and scFv clones specific to PDL1 can be identified from the library following routine procedures. Positive clones can be subjected to further screening to identify those that enhance PDL1 activity.

Antibodies obtained following a method known in the art and described herein can be characterized using methods well known in the art. For example, one method is to identify the epitope to which the antigen binds, or “epitope mapping.” There are many methods known in the art for mapping and characterizing the location of epitopes on proteins, including solving the crystal structure of an antibody-antigen complex, competition assays, gene fragment expression assays, and synthetic peptide-based assays, as described, for example, in Chapter 11 of Harlow and Lane, Using Antibodies, a Laboratory Manual, Cold Spring Harbor Laboratory Press, Cold Spring Harbor, N.Y., 1999. In an additional example, epitope mapping can be used to determine the sequence, to which an antibody binds. The epitope can be a linear epitope, i.e., contained in a single stretch of amino acids, or a conformational epitope formed by a three-dimensional interaction of amino acids that may not necessarily be contained in a single stretch (primary structure linear sequence). Peptides of varying lengths (e.g., at least 4-6 amino acids long) can be isolated or synthesized (e.g., recombinantly) and used for binding assays with an antibody. In another example, the epitope to which the antibody binds can be determined in a systematic screening by using overlapping peptides derived from the target antigen sequence and determining binding by the antibody. According to the gene fragment expression assays, the open reading frame encoding the target antigen is fragmented either randomly or by specific genetic constructions and the reactivity of the expressed fragments of the antigen with the antibody to be tested is determined. The gene fragments may, for example, be produced by PCR and then transcribed and translated into protein in vitro, in the presence of radioactive amino acids. The binding of the antibody to the radioactively labeled antigen fragments is then determined by immunoprecipitation and gel electrophoresis. Certain epitopes can also be identified by using large libraries of random peptide sequences displayed on the surface of phage particles (phage libraries).

Alternatively, a defined library of overlapping peptide fragments can be tested for binding to the test antibody in simple binding assays. In an additional example, mutagenesis of an antigen binding domain, domain swapping experiments and alanine scanning mutagenesis can be performed to identify residues required, sufficient, and/or necessary for epitope binding. For example, domain swapping experiments can be performed using a mutant of a target antigen in which various fragments of PDL1 have been replaced (swapped) with sequences from a closely related, but antigenically distinct protein (such as another member of the tumor necrosis factor receptor family). By assessing binding of the antibody to the mutant PDL1, the importance of the particular antigen fragment to antibody binding can be assessed.

Alternatively, competition assays can be performed using other antibodies known to bind to the same antigen to determine whether an antibody binds to the same epitope as the other antibodies. Competition assays are well known to those of skill in the art.

In some examples, an anti-PDL1 antibody can be prepared by recombinant technology as exemplified below.

Nucleic acids encoding the heavy and light chain of an anti-PDL1 antibody as described herein can be cloned into one expression vector, each nucleotide sequence being in operable linkage to a suitable promoter. In one example, each of the nucleotide sequences encoding the heavy chain and light chain is in operable linkage to a distinct prompter. Alternatively, the nucleotide sequences encoding the heavy chain and the light chain can be in operable linkage with a single promoter, such that both heavy and light chains are expressed from the same promoter. When necessary, an internal ribosomal entry site (IRES) can be inserted between the heavy chain and light chain encoding sequences.

In some examples, the nucleotide sequences encoding the two chains of the antibody are cloned into two vectors, which can be introduced into the same or different cells. When the two chains are expressed in different cells, each of them can be isolated from the host cells expressing such and the isolated heavy chains and light chains can be mixed and incubated under suitable conditions allowing for the formation of the antibody.

Generally, a nucleic acid sequence encoding one or all chains of an antibody can be cloned into a suitable expression vector in operable linkage with a suitable promoter using methods known in the art. For example, the nucleotide sequence and vector can be contacted, under suitable conditions, with a restriction enzyme to create complementary ends on each molecule that can pair with each other and be joined together with a ligase. Alternatively, synthetic nucleic acid linkers can be ligated to the termini of a gene. These synthetic linkers contain nucleic acid sequences that correspond to a particular restriction site in the vector. The selection of expression vectors/promoter would depend on the type of host cells for use in producing the antibodies.

A variety of promoters can be used for expression of the antibodies described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter, and the herpes simplex tk virus promoter.

Regulatable promoters can also be used. Such regulatable promoters include those using the lac repressor from E. coli as a transcription modulator to regulate transcription from lac operator-bearing mammalian cell promoters [Brown, M. et al., Cell, 49:603-612 (1987)], those using the tetracycline repressor (tetR) [Gossen, M., and Bujard, H., Proc. Natl. Acad. Sci. USA 89:5547-5551 (1992); Yao, F. et al., Human Gene Therapy, 9:1939-1950 (1998); Shockelt, P., et al., Proc. Natl. Acad. Sci. USA, 92:6522-6526 (1995)]. Other systems include FK506 dimer, VP16 or p65 using astradiol, RU486, diphenol murislerone, or rapamycin. Inducible systems are available from Invitrogen, Clontech and Ariad.

Regulatable promoters that include a repressor with the operon can be used. In one embodiment, the lac repressor from E. coli can function as a transcriptional modulator to regulate transcription from lac operator-bearing mammalian cell promoters [M. Brown et al., Cell, 49:603-612 (1987); Gossen and Bujard (1992); M. Gossen et al., Natl. Acad. Sci. USA, 89:5547-5551 (1992)] combined the tetracycline repressor (tetR) with the transcription activator (VP 16) to create a tetR-mammalian cell transcription activator fusion protein, tTa (tetR-VP 16), with the tetO-bearing minimal promoter derived from the human cytomegalovirus (hCMV) major immediate-early promoter to create a tetR-tet operator system to control gene expression in mammalian cells. In one embodiment, a tetracycline inducible switch is used. The tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter (Yao et al., Human Gene Therapy, 10(16): 1392-1399 (2003)). One particular advantage of this tetracycline inducible switch is that it does not require the use of a tetracycline repressor-mammalian cells transactivator or repressor fusion protein, which in some instances can be toxic to cells (Gossen et al., Natl. Acad. Sci. USA, 89:5547-5551 (1992); Shockett et al., Proc. Natl. Acad. Sci. USA, 92:6522-6526 (1995)), to achieve its regulatable effects.

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art.

Examples of polyadenylation signals useful to practice the methods described herein include, but are not limited to, human collagen I polyadenylation signal, human collagen II polyadenylation signal, and SV40 polyadenylation signal.

One or more vectors (e.g., expression vectors) comprising nucleic acids encoding any of the antibodies may be introduced into suitable host cells for producing the antibodies. The host cells can be cultured under suitable conditions for expression of the antibody or any polypeptide chain thereof. Such antibodies or polypeptide chains thereof can be recovered by the cultured cells (e.g., from the cells or the culture supernatant) via a conventional method, e.g., affinity purification. If necessary, polypeptide chains of the antibody can be incubated under suitable conditions for a suitable period of time allowing for production of the antibody.

In some embodiments, methods for preparing an antibody described herein involve a recombinant expression vector that encodes both the heavy chain and the light chain of an anti-PDL1 antibody, as also described herein. The recombinant expression vector can be introduced into a suitable host cell (e.g., a dhfr− CHO cell) by a conventional method, e.g., calcium phosphate-mediated transfection. Positive transformant host cells can be selected and cultured under suitable conditions allowing for the expression of the two polypeptide chains that form the antibody, which can be recovered from the cells or from the culture medium. When necessary, the two chains recovered from the host cells can be incubated under suitable conditions allowing for the formation of the antibody.

In one example, two recombinant expression vectors are provided, one encoding the heavy chain of the anti-PDL1 antibody and the other encoding the light chain of the anti-PDL1 antibody. Both of the two recombinant expression vectors can be introduced into a suitable host cell (e.g., dhfr− CHO cell) by a conventional method, e.g., calcium phosphate-mediated transfection. Alternatively, each of the expression vectors can be introduced into a suitable host cells. Positive transformants can be selected and cultured under suitable conditions allowing for the expression of the polypeptide chains of the antibody. When the two expression vectors are introduced into the same host cells, the antibody produced therein can be recovered from the host cells or from the culture medium. If necessary, the polypeptide chains can be recovered from the host cells or from the culture medium and then incubated under suitable conditions allowing for formation of the antibody. When the two expression vectors are introduced into different host cells, each of them can be recovered from the corresponding host cells or from the corresponding culture media. The two polypeptide chains can then be incubated under suitable conditions for formation of the antibody.

Standard molecular biology techniques are used to prepare the recombinant expression vector, transfect the host cells, select for transformants, culture the host cells and recovery of the antibodies from the culture medium. For example, some antibodies can be isolated by affinity chromatography with a Protein A or Protein G coupled matrix.

Any of the nucleic acids encoding the heavy chain, the light chain, or both of an anti-PDL1 antibody as described herein, vectors (e.g., expression vectors) containing such; and host cells comprising the vectors are within the scope of the present disclosure.

### III. Applications of Anti-PDL1 Antibodies

Any of the anti-PDL1 antibodies disclosed herein can be used for therapeutic, diagnostic, and/or research purposes, all of which are within the scope of the present disclosure.

### Pharmaceutical Compositions

The antibodies, as well as the encoding nucleic acids or nucleic acid sets, vectors comprising such, or host cells comprising the vectors, as described herein can be mixed with a pharmaceutically acceptable carrier (excipient) to form a pharmaceutical composition for use in treating a target disease. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover.

The pharmaceutical compositions to be used in the present methods can comprise pharmaceutically acceptable carriers, excipients, or stabilizers in the form of lyophilized formulations or aqueous solutions. (Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

In some examples, the pharmaceutical composition described herein comprises liposomes containing the antibodies (or the encoding nucleic acids) which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The antibodies, or the encoding nucleic acid(s), may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are known in the art, see, e.g., Remington, The Science and Practice of Pharmacy 20th Ed. Mack Publishing (2000).

In other examples, the pharmaceutical composition described herein can be formulated in sustained-release format. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The pharmaceutical compositions to be used for in vivo administration must be sterile. This is readily accomplished by, for example, filtration through sterile filtration membranes. Therapeutic antibody compositions are generally placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g., Tween™ 20, 40, 60, 80 or 85) and other sorbitans (e.g., Span™ 20, 40, 60, 80 or 85). Compositions with a surface-active agent will conveniently comprise between 0.05 and 5% surface-active agent and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g., egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets between 0.1 and 1.0 μm, particularly 0.1 and 0.5 μm, and have a pH in the range of 5.5 to 8.0.

The emulsion compositions can be those prepared by mixing an antibody with Intralipid™ or the components thereof (soybean oil, egg phospholipids, glycerol and water).

### Therapeutic Applications

To practice the method disclosed herein, an effective amount of the pharmaceutical composition described herein can be administered to a subject (e.g., a human) in need of the treatment via a suitable route, such as intravenous administration, e.g., as a bolus or by continuous infusion over a period of time, by intramuscular, intraperitoneal, intracerebrospinal, subcutaneous, intra-articular, intrasynovial, intrathecal, oral, inhalation or topical routes. Commercially available nebulizers for liquid formulations, including jet nebulizers and ultrasonic nebulizers are useful for administration. Liquid formulations can be directly nebulized and lyophilized powder can be nebulized after reconstitution. Alternatively, the antibodies as described herein can be aerosolized using a fluorocarbon formulation and a metered dose inhaler, or inhaled as a lyophilized and milled powder.

The subject to be treated by the methods described herein can be a mammal, more preferably a human. Mammals include, but are not limited to, farm animals, sport animals, pets, primates, horses, dogs, cats, mice and rats. A human subject who needs the treatment may be a human patient having, at risk for, or suspected of having a target disease/disorder characterized by carrying PDL1⁺ disease cells. Examples of such target diseases/disorders include hematopoietic cancers, e.g., a cancer of B cell lineage. Examples include, but are not limited to, hematological B cell neoplasms including lymphocytic leukemia, e.g., B Cell chronic lymphocytic leukemia (CLL); B-cell acute lymphoblastic leukemia (ALL), and B-cell non-Hodgkin's lymphoma (NHL).

A subject having a target cancer can be identified by routine medical examination, e.g., laboratory tests, organ functional tests, CT scans, or ultrasounds. In some embodiments, the subject to be treated by the method described herein may be a human cancer patient who has undergone or is subjecting to an anti-cancer therapy, for example, chemotherapy, radiotherapy, immunotherapy, or surgery.

A subject suspected of having any of such target disease/disorder might show one or more symptoms of the disease/disorder. A subject at risk for the disease/disorder can be a subject having one or more of the risk factors for that disease/disorder.

As used herein, “an effective amount” refers to the amount of each active agent required to confer therapeutic effect on the subject, either alone or in combination with one or more other active agents. Determination of whether an amount of the antibody achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of a target disease/disorder. Alternatively, sustained continuous release formulations of an antibody may be appropriate. Various formulations and devices for achieving sustained release are known in the art.

In one example, dosages for an antibody as described herein may be determined empirically in individuals who have been given one or more administration(s) of the antibody. Individuals are given incremental dosages of the agonist. To assess efficacy of the agonist, an indicator of the disease/disorder can be followed.

For the purpose of the present disclosure, a typical daily dosage might range from about any of 0.1 μg/kg to 3 μg/kg to 30 μg/kg to 300 μg/kg to 3 mg/kg, to 30 mg/kg to 100 mg/kg or more, depending on the factors mentioned above. For repeated administrations over several days or longer, depending on the condition, the treatment is sustained until a desired suppression of symptoms occurs or until sufficient therapeutic levels are achieved to alleviate a target disease or disorder, or a symptom thereof. The progress of this therapy is easily monitored by conventional techniques and assays. The dosing regimen (including the antibody used) can vary over time.

The particular dosage regimen, i.e., dose, timing and repetition, will depend on the particular individual and that individual's medical history, as well as the properties of the individual agents (such as the half-life of the agent, and other considerations well known in the art).

For the purpose of the present disclosure, the appropriate dosage of an antibody as described herein will depend on the specific antibody, antibodies, and/or non-antibody peptide (or compositions thereof) employed, the type and severity of the disease/disorder, whether the antibody is administered for preventive or therapeutic purposes, previous therapy, the patient's clinical history and response to the agonist, and the discretion of the attending physician. Typically, the clinician will administer an antibody, until a dosage is reached that achieves the desired result. In some embodiments, the desired result is an increase in anti-tumor immune response in the tumor microenvironment. Methods of determining whether a dosage resulted in the desired result would be evident to one of skill in the art. Administration of one or more antibodies can be continuous or intermittent, depending, for example, upon the recipient's physiological condition, whether the purpose of the administration is therapeutic or prophylactic, and other factors known to skilled practitioners. The administration of an antibody may be essentially continuous over a preselected period of time or may be in a series of spaced dose, e.g., either before, during, or after developing a target disease or disorder.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease or reducing disease severity or prolonging survival. Alleviating the disease or prolonging survival does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

Conventional methods, known to those of ordinary skill in the art of medicine, can be used to administer the pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot routes of administration such as using 1-, 3-, or 6-month depot injectable or biodegradable materials and methods. In some examples, the pharmaceutical composition is administered intraocularly or intravitreally.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipient is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

In one embodiment, an antibody is administered via site-specific or targeted local delivery techniques. Examples of site-specific or targeted local delivery techniques include various implantable depot sources of the antibody or local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication No. WO 00/53211 and U.S. Pat. No. 5,981,568.

Targeted delivery of therapeutic compositions containing an antisense polynucleotide, expression vector, or subgenomic polynucleotides can also be used. Receptor-mediated DNA delivery techniques are described in, for example, Findeis et al., Trends Biotechnol. (1993) 11:202; Chiou et al., Gene Therapeutics: Methods And Applications Of Direct Gene Transfer (J. A. Wolff, ed.) (1994); Wu et al., J. Biol. Chem. (1988) 263:621; Wu et al., J. Biol. Chem. (1994) 269:542; Zenke et al., Proc. Natl. Acad. Sci. USA (1990) 87:3655; Wu et al., J. Biol. Chem. (1991) 266:338.

Therapeutic compositions containing a polynucleotide (e.g., those encoding the antibodies described herein) are administered in a range of about 100 ng to about 200 mg of DNA for local administration in a gene therapy protocol. In some embodiments, concentration ranges of about 500 ng to about 50 mg, about 1 μg to about 2 mg, about 5 μg to about 500 μg, and about 20 μg to about 100 μg of DNA or more can also be used during a gene therapy protocol.

The therapeutic polynucleotides and polypeptides described herein can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters and/or enhancers. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740 and 4,777,127; GB Patent No. 2,200,651; and EP Patent No. 0 345 242), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., PCT Publication Nos. WO 94/12649, WO 93/03769; WO 93/19191; WO 94/28938; WO 95/11984 and WO 95/00655). Administration of DNA linked to killed adenovirus as described in Curiel, Hum. Gene Ther. (1992) 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther. (1992) 3:147); ligand-linked DNA (see, e.g., Wu, J. Biol. Chem. (1989) 264:16985); eukaryotic cell delivery vehicles cells (see, e.g., U.S. Pat. No. 5,814,482; PCT Publication Nos. WO 95/07994; WO 96/17072; WO 95/30763; and WO 97/42338) and nucleic charge neutralization or fusion with cell membranes. Naked DNA can also be employed. Exemplary naked DNA introduction methods are described in PCT Publication No. WO 90/11092 and U.S. Pat. No. 5,580,859. Liposomes that can act as gene delivery vehicles are described in U.S. Pat. No. 5,422,120; PCT Publication Nos. WO 95/13796; WO 94/23697; WO 91/14445; and EP Patent No. 0524968. Additional approaches are described in Philip, Mol. Cell. Biol. (1994) 14:2411, and in Woffendin, Proc. Natl. Acad. Sci. (1994) 91:1581.

The particular dosage regimen, i.e., dose, timing and repetition, used in the method described herein will depend on the particular subject and that subject's medical history.

In some embodiments, more than one antibody, or a combination of an antibody and another suitable therapeutic agent, may be administered to a subject in need of the treatment. The antibody can also be used in conjunction with other agents that serve to enhance and/or complement the effectiveness of the agents.

Treatment efficacy for a target disease/disorder can be assessed by methods well-known in the art.

### Diagnostic Applications

Any of the anti-PDL1 antibodies disclosed here may be used for detecting and quantifying PDL1 levels or PDL1+ cell levels in a biological sample using a conventional method, for example, any immunohistological method known to those of skill in the art (see, e.g., Jalkanen, et al., J. Cell. Biol. 101:976-985 (1985); Jalkanen et al., J. Cell Biol. 105:3087-3096 (1987)). Other antibody-based methods useful for detecting PDL1 expression include immunoassays, such as the enzyme linked immunosorbent assay (ELISA), immunoprecipitation, or Western blotting. Suitable assays are described in more detail elsewhere herein.

The term “biological sample” means any biological sample obtained from an individual, cell line, tissue culture, or other source of cells potentially expressing PDL1 Methods for obtaining tissue biopsies and body fluids from mammals are well known in the art.

To perform the method disclosed herein, any of the anti-PDL1 antibodies as disclosed herein can be brought in contact with a sample suspected of containing a target antigen as disclosed herein, for example, a human PDL1 protein or a PDL1+ cell. In general, the term “contacting” or “in contact” refers to an exposure of the anti-PDL1 antibody disclosed herein with the sample suspected of containing the target antigen for a suitable period under suitable conditions sufficient for the formation of a complex between the anti-PDL1 antibody and the target antigen in the sample, if any. The antibody-antigen complex thus formed, if any, can be determined via a routine approach. Detection of such an antibody-antigen complex after the incubation is indicative of the presence of the target antigen in the sample. When needed, the amount of the antibody-antigen complex can be quantified, which is indicative of the level of the target antigen in the sample.

In some examples, the anti-PDL1 antibodies as described herein can be conjugated to a detectable label, which can be any agent capable of releasing a detectable signal directly or indirectly. The presence of such a detectable signal or intensity of the signal is indicative of presence or quantity of the target antigen in the sample. Alternatively, a secondary antibody specific to the anti-PDL1 antibody or specific to the target antigen may be used in the methods disclosed herein. For example, when the anti-PDL1 antibody used in the method is a full-length antibody, the secondary antibody may bind to the constant region of the anti-PDL1 antibody. In other instances, the secondary antibody may bind to an epitope of the target antigen that is different from the binding epitope of the anti-PDL1 antibody. Any of the secondary antibodies disclosed herein may be conjugated to a detectable label.

Any suitable detectable label known in the art can be used in the assay methods described herein. In some embodiments, a detectable label can be a label that directly releases a detectable signal. Examples include a fluorescent label or a dye. A fluorescent label comprises a fluorophore, which is a fluorescent chemical compound that can re-emit light upon light excitation. Examples of fluorescent label include, but are not limited to, xanthene derivatives (e.g., fluorescein, rhodamine, Oregon green, eosin, and Texas red), cyanine derivatives (e.g., cyanine, indocarbocyanine, oxacarbocyanine, thiacarbocyanine, and merocyanine), squaraine derivatives and ring-substituted squaraines (e.g., Seta and Square dyes), squaraine rotaxane derivatives such as SeTau dyes, naphthalene derivatives (e.g., dansyl and prodan derivatives), coumarin derivatives, oxadiazole derivatives (e.g., pyridyloxazole, nitrobenzoxadiazole and benzoxadiazole), anthracene derivatives (e.g., anthraquinones, including DRAQ5, DRAQ7 and CyTRAK Orange), pyrene derivatives such as cascade blue, oxazine derivatives (e.g., Nile red, Nile blue, cresyl violet, and oxazine 170), acridine derivatives (e.g., proflavin, acridine orange, and acridine yellow), arylmethine derivatives (e.g., auramine, crystal violet, and malachite green), and tetrapyrrole derivatives (e.g., porphin, phthalocyanine, and bilirubin). A dye can be a molecule comprising a chromophore, which is responsible for the color of the dye. In some examples, the detectable label can be fluorescein isothiocyanate (FITC), phycoerythrin (PE), biotin, Allophycocyanin (APC) or Alexa Fluor® 488.

In some embodiments, the detectable label may be a molecule that releases a detectable signal indirectly, for example, via conversion of a reagent to a product that directly releases the detectable signal. In some examples, such a detectable label may be an enzyme (e.g., β-galactosidase, HRP or AP) capable of producing a colored product from a colorless substrate.

### Kits for Use in Treatment of Diseases

The present disclosure also provides kits for use in treating or alleviating a target disease, such as hematopoietic cancer as described herein. Such kits can include one or more containers comprising an anti-PDL1 antibody, e.g., any of those described herein. In some instances, the anti-PDL1 antibody may be co-used with a second therapeutic agent.

In some embodiments, the kit can comprise instructions for use in accordance with any of the methods described herein. The included instructions can comprise a description of administration of the anti-PDL1 antibody, and optionally the second therapeutic agent, to treat, delay the onset, or alleviate a target disease as those described herein. The kit may further comprise a description of selecting an individual suitable for treatment based on identifying whether that individual has the target disease, e.g., applying the diagnostic method as described herein. In still other embodiments, the instructions comprise a description of administering an antibody to an individual at risk of the target disease.

The instructions relating to the use of an anti-PDL1 antibody generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, delaying the onset and/or alleviating the disease, such as cancer or immune disorders (e.g., an autoimmune disease). Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an anti-PDL1 antibody as those described herein.

Kits may optionally provide additional components such as buffers and interpretive information. Normally, the kit comprises a container and a label or package insert(s) on or associated with the container. In some embodiments, the invention provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R.I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1: Production of High Affinity Anti-PDL1 Antibodies

This example discloses production of exemplary anti-PDL1 antibodies with high binding affinity to human PDL1.

### (i) Generation of 12A4 Fab Display Library

A phagemid pSY6 displaying the antibody Fab VL gene between NsiI and KpnI sites was double digested with NsiI and KpnI, gel purified, and ligated to 12A4 VL gene insert with proper double digestion to generate pSY6_12 A4VL. Phagemid pSY1_12 A4VL was then sequentially digested with PspOMI and BglII, gel purified to remove the generic VH gene insert, and ligated to 12A4 VH gene insert with proper restriction enzyme digestion to generate pSY9 phagemid. Uracil containing single stranded DNA template was then generated with a procedure similar to that described (Sidhu S. S et al Methods Enzymol 328, 333-63 (2000)).

(ii) Construction of the pSY9_CDRW_L1_L2_L3 Library

An antibody library allowing for screening of CDRs L1, L2 and L3 at the same time was constructed as follows. A single amino acid CDR walking strategy (Yang W. P., et al J. Mol. Biol. 254, 392-403 (1995)) was adopted. The randomized positions included 24-25 and 27-34 in CDR-L1, positions 50-56 in CDR-L2, and positions 91-97 in CDR-L3. Each position was randomized individually at a given CDR with the degenerate codon NNS to encode all 20 amino acids. Randomization was incorporated into pSY9 ssDNA template via Kunkel mutagenesis (Kunkel T. A. Proc Natl Acad Sci USA 82, 488-92 (1985); Sidhu S. S et al Methods Enzymol 328, 333-63 (2000)). Phosphorylation of the primers and Kunkel mutagenesis were carried out as described (Sidhu S. S et al Methods Enzymol 328, 333-63 (2000)). Covalently closed circular DNA obtained was electroporated into electrocompetent SS320 cells to prepare pSY9_CDRW_L1_L2_L3 library as described (Sidhu S. S et al Methods Enzymol 328, 333-63 (2000)). The tittered apparent diversity was 1.31×10¹⁰, larger than 1.8×10⁷, the designed theoretical diversity.

(iii) Construction of the pSY9_CDRW_H1_H2_H3 Library

An antibody library allowing for screening of CDRs H1, H2 and H3 at the same time was constructed as follows. A single amino acid CDR walking strategy (Yang W. P., et al J. Mol. Biol. 254, 392-403 (1995)) was adopted. The randomized positions included 27-35 in CDR-H1, positions 50 and 52-58 in CDR-H2, and positions 95-102 in CDR-H3. Each position was randomized individually at a given CDR with the degenerate codon NNS to encode all 20 amino acids. Randomization was incorporated into pSY9 ssDNA template via Kunkel mutagenesis (Kunkel T. A. Proc Natl Acad Sci USA 82, 488-92 (1985); Sidhu S. S et al Methods Enzymol 328, 333-63 (2000)). Phosphorylation of the primers and Kunkel mutagenesis were carried out as described (Sidhu S. S et al Methods Enzymol 328, 333-63 (2000)). Covalently closed circular DNA obtained was electroporated into electrocompetent SS320 cells to prepare pSY9_CDRW_H1_H2_H3 library as described (Sidhu S. S et al Methods Enzymol 328, 333-63 (2000)). The tittered apparent diversity was 1.38×10¹⁰, larger than 3.7×10⁷, the designed theoretical diversity.

### (iv) Selection of Affinity Maturated Antibodies

Selection was carried out similar to that have been described previously (Ye J. D., et al Proc Natl Acad Sci USA 105, 82-87 (2008)). Biotinylated N-terminal segment (Met1-Thr239) of the extracellular domain of human PD-L1 was used as the antigen. In each round except for the first round, purified phage pools were first incubated with streptavidin beads for 15 min, and the supernatant was used in the subsequent selection on a KingFisher magnetic particle processor (Thermo Fisher). Phages (10¹⁰⁻¹¹ cfu) were incubated for 15 min-1 hr with biotinylated PD-L1 (10, 4, 2, 0.5 and 0.5 nM for round 1-5, respectively). Streptavidin magnetic beads were then added to the solution for 1.5-15 min to allow the capture of the biotinylated PDL1 together with the bound phages. In the fourth round, after capture of the antigen/antibody complex on the beads, the beads were washed with PBS supplemented with 0.05% Tween® 20 (PT) and >1000 fold of non-biotinylated PD-L1 was incubated with the beads for 1 hour at RT. The beads were then washed five times with PT, and eluted in 100 mM DTT for 15 min. After each round of selection, recovered phages were amplified as described (Sidhu S. S et al Methods Enzymol 328, 333-63 (2000)).

(v) Phage ELISA Screening with Low Antigen Concentration (PESLA)

After 3-5 rounds of selection, individual clones were analyzed by phage ELISA (Ye J. D., et al Proc Natl Acad Sci USA 105, 82-87 (2008)). The procedure was modified to screen high affinity binders in a high throughput manner. Forty-eight or more individual colonies were picked from a fresh LB/Amp plate, inoculated in 300 μL of 2YT medium containing 100 μg/mL ampicillin and 10¹⁰ PFU/mL M13KO7 helper phage in a 96-well deep-well plate, and grown at 37° C. overnight with shaking at 300 rpm. The deep-well plate was then centrifuged for 15 min at 3500 rpm and 4° C. to pellet the cells. The supernatant was diluted 3-fold with phage dilution buffer (PBS, 0.5% BSA, and 0.05% Tween 20) to prepare a phage solution. A 96-well Maxisorp plate was coated with 100 μL of 0.1 μg/mL PD-L1 in 100 mM sodium bicarbonate coating buffer (pH 9.6) overnight at 4° C. The coating solution was removed and the Maxisorp plate was blocked for 1 h with 200 μL/well of 1% (w/v) BSA in PBS. After the blocking solution was removed, the Maxisorp plate was washed 4 times with PBS with 0.05% (v/v) Tween 20 (PT) and incubated with 100 μL/well of phage solution for 1 h at room temperature. After washing with PT Buffer 6 times, the Maxisorp plate was incubated with 100 μL/well anti-M13/horseradish peroxidase conjugate (diluted 3000× in phage dilution buffer) at room temperature for 30 min. After washing 6 times with PT Buffer, the Maxisorp plate was incubated with 100 μL/well Ultra TMB-ELISA Substrates for 5-10 min, quenched with 100 μL/well of 1 M phosphoric acid, and read spectrophotometrically at 450 nm in a microplate reader. As the low antigen concentration serves as the limiting factor, higher signals correlates with the tighter binding clones. These tighter binders were miniprepped and sequenced to identify unique clones.

(vi) Characterize the Unique Clones with Competitive Phage ELISA

Six unique clones (12A4a, 12A4e, 12A4f, 12A4g, 12A4h, and 12A4i) and 3 unique clones (12A4b, 12A4c, and 12A4d) were identified from library pSY9_CDRW_L1_L2_L3 and pSY9_CDRW_H1_H2_H3, respectively. Their sequence alignments are provided below (mutations relative to the parent antibody are underlined):

| Light Chain Variable Region Sequence Alignment (Sequences from | |
|---|---|
| top to bottom are SEQ ID NOs: 50-63) | |
| 12A4_VL | EIVLTQSPATLSLSPGERATLSCRASQSVSSYLAWYQQKPGQAPRLLIYDASNRAT |
| 12A4a_VL | EIVLTQSPATLSLSPGERATLSCRASQSVSSYLAWYQQKPGQAPRLLIYDASNRAP |
| 12A4b_VL | EIVLTQSPATLSLSPGERATLSCRASQSVSSYLAWYQQKPGQAPRLLIYDASNRAT |
| 12A4c_VL | EIVLTQSPATLSLSPGERATLSCRASQSVSSYLAWYQQKPGQAPRLLIYDASNRAT |
| 12A4d_VL | EIVLTQSPATLSLSPGERATLSCRASQSVSSYLAWYQQKPGQAPRLLIYDASNRAT |
| 12A4e_VL | EIVLTQSPATLSLSPGERATLSCRASQSVSSYLAWYQQKPGQAPRLLIYDASNRAT |
| 12A4f_VL | EIVLTQSPATLSLSPGERATLSCRASQSVSSYLAWYQQKPGQAPRLLIYDASNRAM |
| 12A4g_VL | EIVLTQSPATLSLSPGERATLSCRASQSVSSYLSWYQQKPGQAPRLLIYDASNRAP |
| 12A4h_VL | EIVLTQSPATLSLSPGERATLSCRASQSVSSYMAWYQQKPGQAPRLLIYDASNRAE |
| 12A4i_VL | EIVLTQSPATLSLSPGERATLSCRASQSVSSYLEWYQQKPGQAPRLLIYDASNRAT |
| 12A4ad_VL | EIVLTQSPATLSLSPGERATLSCRASQSVSSYLAWYQQKPGQAPRLLIYDASNRAP |
| 12A4ba_VL | EIVLTQSPATLSLSPGERATLSCRASQSVSSYLAWYQQKPGQAPRLLIYDASNRAP |
| 12A4bd_VL | EIVLTQSPATLSLSPGERATLSCRASQSVSSYLAWYQQKPGQAPRLLIYDASNRAT |
| 12A4bad_VL | EIVLTQSPATLSLSPGERATLSCRASQSVSSYLAWYQQKPGQAPRLLIYDASNRAP |
| 12A4_VL | GIPARFSGSGSGTDFTLTISSLEPEDFAVYYCQQRSNWPTFGQGTKVEIK |
| 12A4a_VL | GIPARFSGSGSGTDFTLTISSLEPEDFAVYYCQQRSNWPTFGQGTKVEIK |
| 12A4b_VL | GIPARFSGSGSGTDFTLTISSLEPEDFAVYYCQQRSNWPTFGQGTKVEIK |
| 12A4c_VL | GIPARFSGSGSGTDFTLTISSLEPEDFAVYYCQQRSNWPTFGQGTKVEIK |
| 12A4d_VL | GIPARFSGSGSGTDFTLTISSLEPEDFAVYYCQQRSNWPTFGQGTKVEIK |
| 12A4e_VL | GIPARFSGSGSGTDFTLTISSLEPEDFAVYYCQQRANWPTFGQGTKVEIK |
| 12A4f_VL | GIPARFSGSGSGTDFTLTISSLEPEDFAVYYCQQRSNWPTFGQGTKVEIK |
| 12A4g_VL | GIPARFSGSGSGTDFTLTISSLEPEDFAVYYCQQRSNWPTFGQGTKVEIK |
| 12A4h_VL | GIPARFSGSGSGTDFTLTISSLEPEDFAVYYCQQRANWPTFGQGTKVEIK |
| 12A4i_VL | GIPARFSGSGSGTDFTLTISSLEPEDFAVYYCQQRSNWPTFGQGTKVEIK |
| 12A4ad_VL | GIPARFSGSGSGTDFTLTISSLEPEDFAVYYCQQRSNWPTFGQGTKVEIK |
| 12A4ba_VL | GIPARFSGSGSGTDFTLTISSLEPEDFAVYYCQQRSNWPTFGQGTKVEIK |
| 12A4bd_VL | GIPARFSGSGSGTDFTLTISSLEPEDFAVYYCQQRSNWPTFGQGTKVEIK |
| 12A4bad_VL | GIPARFSGSGSGTDFTLTISSLEPEDFAVYYCQQRSNWPTFGQGTKVEIK |
| Heavy Chain Variable Region Sequence Alignment (Sequences from | |
| top to bottom are SEQ ID NOs: 64-77) | |
| 12A4_VH | QVQLVQSGAEVKKPGSSVKVSCKTSGDTFSTYAISWVRQAPGQGLEWMGGIIPIFGKAH |
| 12A4a_VH | QVQLVQSGAEVKKPGSSVKVSCKTSGDTFSTYAISWVRQAPGQGLEWMGGIIPIFGKAH |
| 12A4b_VH | QVQLVQSGAEVKKPGSSVKVSCKTSGDTFSTYAISWVRQAPGQGLEWMGGIIPIFGKAH |
| 12A4c_VH | QVQLVQSGAEVKKPGSSVKVSCKTSGDTFSTYAISWVRQAPGQGLEWMGGIIPIFGKAH |
| 12A4d_VH | QVQLVQSGAEVKKPGSSVKVSCKTSGDTFSTYAISWVRQAPGQGLEWMGGIIPLFGKAH |
| 12A4e_VH | QVQLVQSGAEVKKPGSSVKVSCKTSGDTFSTYAISWVRQAPGQGLEWMGGIIPIFGKAH |
| 12A4f_VH | QVQLVQSGAEVKKPGSSVKVSCKTSGDTFSTYAISWVRQAPGQGLEWMGGIIPIFGKAH |
| 12A4g_VH | QVQLVQSGAEVKKPGSSVKVSCKTSGDTFSTYAISWVRQAPGQGLEWMGGIIPIFGKAH |
| 12A4h_VH | QVQLVQSGAEVKKPGSSVKVSCKTSGDTFSTYAISWVRQAPGQGLEWMGGIIPIFGKAH |
| 12A4i_VH | QVQLVQSGAEVKKPGSSVKVSCKTSGDTFSTYAISWVRQAPGQGLEWMGGIIPIFGKAH |
| 12A4ad_VH | QVQLVQSGAEVKKPGSSVKVSCKTSGDTFSTYAISWVRQAPGQGLEWMGGIIPLFGKAH |
| 12A4ba_VH | QVQLVQSGAEVKKPGSSVKVSCKTSGDTFSTYAISWVRQAPGQGLEWMGGIIPIFGKAH |
| 12A4bd_VH | QVQLVQSGAEVKKPGSSVKVSCKTSGDTFSTYAISWVRQAPGQGLEWMGGIIPLFGKAH |
| 12A4bad_VH | QVQLVQSGAEVKKPGSSVKVSCKTSGDTFSTYAISWVRQAPGQGLEWMGGIIPLFGKAH |
| 12A4_VH | YAQKFQGRVTITADESTSTAYMELSSLRSEDTAVYFCARKFHFVSGSPFGMDVWGQGTT |
| 12A4a_VH | YAQKFQGRVTITADESTSTAYMELSSLRSEDTAVYFCARKFHFVSGSPFGMDVWGQGTT |
| 12A4b_VH | YAQKFQGRVTITADESTSTAYMELSSLRSEDTAVYFCARKFHFVRGSPFGMDVWGQGTT |
| 12A4c_VH | YAQKFQGRVTITADESTSTAYMELSSLRSEDTAVYFCARKFRFVSGSPFGMDVWGQGTT |
| 12A4d_VH | YAQKFQGRVTITADESTSTAYMELSSLRSEDTAVYFCARKFHFVSGSPFGMDVWGQGTT |
| 12A4e_VH | YAQKFQGRVTITADESTSTAYMELSSLRSEDTAVYFCARKFHFVSGSPFGMDVWGQGTT |
| 12A4f_VH | YAQKFQGRVTITADESTSTAYMELSSLRSEDTAVYFCARKFHFVSGSPFGMDVWGQGTT |
| 12A4g_VH | YAQKFQGRVTITADESTSTAYMELSSLRSEDTAVYFCARKFHFVSGSPFGMDVWGQGTT |
| 12A4h_VH | YAQKFQGRVTITADESTSTAYMELSSLRSEDTAVYFCARKFHFVSGSPFGMDVWGQGTT |
| 12A4i_VH | YAQKFQGRVTITADESTSTAYMELSSLRSEDTAVYFCARKFHFVSGSPFGMDVWGQGTT |
| 12A4ad_VH | YAQKFQGRVTITADESTSTAYMELSSLRSEDTAVYFCARKFHFVSGSPFGMDVWGQGTT |
| 12A4ba_VH | YAQKFQGRVTITADESTSTAYMELSSLRSEDTAVYFCARKFHFVRGSPFGMDVWGQGTT |
| 12A4bd_VH | YAQKFQGRVTITADESTSTAYMELSSLRSEDTAVYFCARKFHFVRGSPFGMDVWGQGTT |
| 12A4bad_VH | YAQKFQGRVTITADESTSTAYMELSSLRSEDTAVYFCARKFHFVRGSPFGMDVWGQGTT |
| 12A4_VH | VTVSS |
| 12A4a_VH | VTVSS |
| 12A4b_VH | VTVSS |
| 12A4c_VH | VTVSS |
| 12A4d_VH | VTVSS |
| 12A4e_VH | VTVSS |
| 12A4f_VH | VTVSS |
| 12A4g_VH | VTVSS |
| 12A4h_VH | VTVSS |
| 12A4i_VH | VTVSS |
| 12A4ad_VH | VTVSS |
| 12A4ba_VH | VTVSS |
| 12A4bd_VH | VTVSS |
| 12A4bad_VH | VTVSS |

Competitive phage ELISA was used to characterize the binding affinities of these clones. MaxiSorp ELISA plate was coated with PD-L1 overnight at 4° C. and blocked with BSA for 1 hr at RT. Serial dilutions of PD-L1 were incubated with subsaturating concentrations of phage at RT for 1 hr, and then added to the blocked and washed ELISA plate. After 15 min incubation and washing, anti-M13 antibody/HRP conjugate was added and incubated for 30 min, then developed with TMB for 5-10 min and quenched with 1 M phosphoric acid. Binding signal was analyzed by the plate reader. Clones 12A4a, 12A4b and 12A4d showed improved binding toward PD-L1 (Table 2 below).

(vii) Combining Beneficial Mutations from Different CDRs

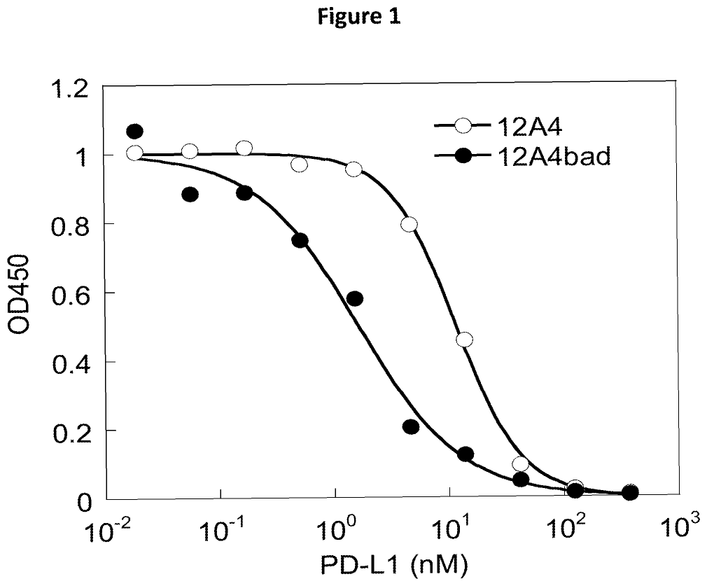

To test whether these mutations have additive effect, four new clones (12A4ad, 12A4ba, 12A4bd, and 12A4bad) were prepared via Kunkel mutagenesis. Their binding affinities to PD-L1 were measured with competitive phage ELISA. Results showed that 12A4bad binds PD-L1 with the highest affinity. See Table 2 below. Exemplar plot of the competitive phage ELISA is shown in FIG. 1.

TABLE 2
Binding affinities of exemplary affinity matured
clones measured by competitive phage ELISA

| Clones | Fab Kd (nM) | Improvement from 12A4 Parent Clone |
|---|---|---|
| 12A4 | 11.2 | 1 |
| 12A4a | 8.3 | 1.3 |
| 12A4b | 3.4 | 3.3 |
| 12A4d | 4.7 | 2.4 |
| 12A4ad | 5.0 | 2.2 |
| 12A4ba | 2.1 | 5.3 |
| 12A4bd | 1.8 | 6.3 |
| 12A4bad | 1.5 | 7.5 |

Unexpectedly, multiple variants (e.g., those listed in Table 2 above) showed better binding activity to PD-L1 as compared with the parent antibody 12A4.

### Example 2. Characterization of Exemplary Anti-PDL1 Monoclonal Antibody 12A4bad

### (i) Binding Affinity to PD-L1-Expressing Tumor Cells

PDL1 is expressed in a large range of tumor cells, including lung cancer, breast cancer, melanoma, etc. MDA MB-231 is a breast cancer cell line which a high expression level of PDL1 and shows the strong immunosuppressive effect to T cells. This experiment investigated the binding affinity of exemplary anti-PDL1 monoclonal antibody 12A4bad to MDA MB-231 cells.

Cultured MDA MB-231 cells were treated with Accutase and collected for binding assay. The cell pellet was resuspended and dissociated as a single cell solution. 1×10⁵ MDAMB-231 cells were plated in 96 well plate for antibody binding. Antibody 12A4bad and durvalumab were serially diluted in the cell staining buffer and incubated at RT for 30 min. After Fluorescent labelled anti-human IgG Fc secondary antibody staining, the samples were analyzed using Accuri C6 cytometer analyzer (BD Biosciences).

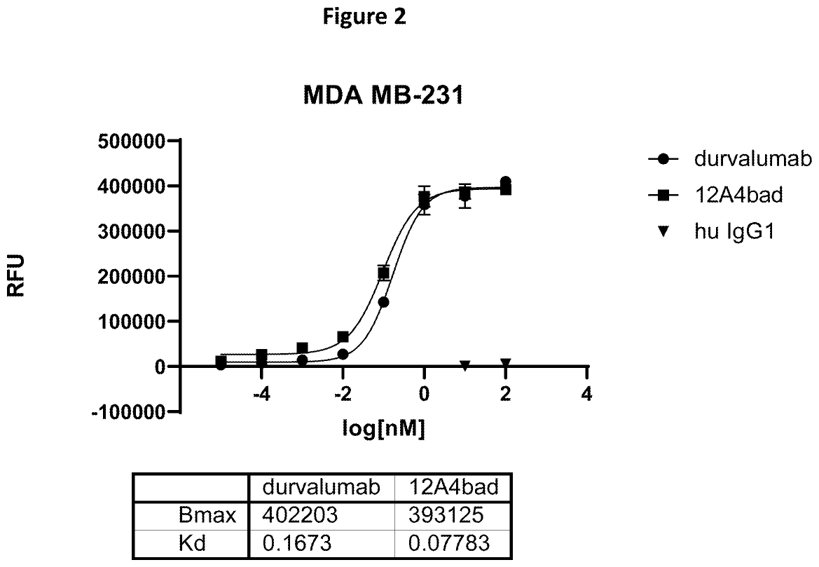

As shown in FIG. 2, the Kd of anti-PDL1 antibody 12A4bad is 0.07783 nM to MDA MB-231 cells, which is lower than that of durvalumab (Kd=0.1673 nM). The control human IgG1 didn't show any binding at concentrations of 10 nM and 100 nM.

### (ii) Blockade of PD1/PD-L1 Interaction

PD1 is an immune inhibitory receptor expressed on activated T cells and B cells and plays a critical role in regulating immune responses. Engagement of PD1 by its ligand PDL1 on an adjacent cell inhibits TCR signaling and TCR-mediated proliferation, transcriptional activation and cytokine production. The PDL1/PD1 blockage assay consists of two genetically engineered cell lines, PD1 effector cells and PDL1 aAPC/CHOK1 cells. When cocultured, the PD1/PDL1 interaction inhibits TCR-mediated luminescence. When the PDL1/PD1 interaction is disrupted, TCR activation induces luminescence (via activation of the NFAT pathway) that can be detected by addition of Bio-Glo reagent and quantitation with a luminometer.

This experiment investigated the blockade activity of exemplary anti-PDL1 antibody 12A4bad against PD1/PD-L1 interaction. Briefly, 4×10⁴ PDL1 aAPC/CHOK1 cells were plated in 96 well plate and cultured overnight. Antibody 12A4bad and durvalumab were serially diluted in assay buffer (RPMI1640/1% FBS). The culture medium was then replaced with 40ul diluted antibody buffer and incubated at RT for 30 min. The PD1 effector cells were thawed and 6×10⁴ cells (in 40ul) were added to each well with different diluted antibodies. The plate was kept in a cell culture incubator for 6 hrs. Afterwards, 80ul freshly prepared Bio-Glo reagent were added to each well. After 15 min reaction in RT with 500 rpm shaking, the luminescent signals were recorded using Bio-Tek Syringy4 plate reader.

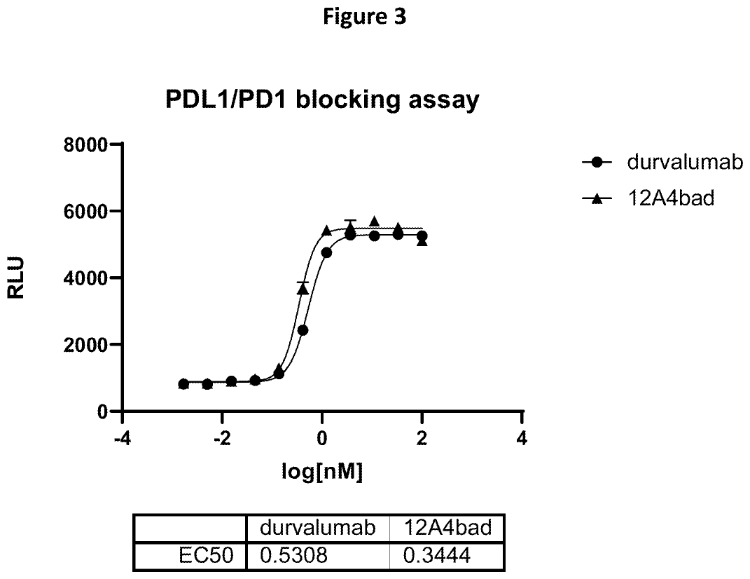

As shown in FIG. 3, both antibodies effectively blocked PDL1-PD1 binding and generate luminescent signal in PD-1 effector cells. The EC₅₀ of PDL1 antibody 12A4bad is 0.3444 nM, lower than that of durvalumab (EC₅₀=0.5308 nM).

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. An antibody that binds human programmed death-ligand 1 (PDL1), wherein the antibody comprises:
(a) a heavy chain variable region comprising
(i) a heavy chain complementary determining region (CDR) 1 that comprises the amino acid sequence of GDTFSTYAIS (SEQ ID NO:1),
(ii) a heavy chain CDR2 that comprises the amino acid sequence of GIIPX₁FGKAH (SEQ ID NO: 2), in which X₁ is I or L, and
(iii) a heavy chain CDR3 that comprises the amino acid sequence of KFX₂FVX₃GSPFGMDV (SEQ ID NO: 5), in which X₂ is H or R and X₃ is S or R; and
(b) a light chain variable region comprising
(i) a light chain CDR1 that comprises the amino acid sequence of RASQSVSSYX₄X₅ (SEQ ID NO: 9), in which X₄ is L or M and X₅ is A, S, or E,
(ii) a light chain CDR2 that comprises the amino acid sequence of DASNRAX₆ (SEQ ID NO: 14), in which X₆ is T, P, M, or E, and
(iii) a light chain CDR3 that comprises the amino acid sequence of QQRX-NWPT (SEQ ID NO: 19), in which X₇ is S or A;
provided that the antibody is not 12A4.

2. The antibody of claim 1, wherein the heavy chain CDR2 is GIIPIFGKAHYAQKFQG (SEQ ID NO:3) or GIIPLFGKAHYAQKFQG (SEQ ID NO: 4).

3. The antibody of claim 1, wherein the heavy chain CDR3 is KFHFVSGSPFGMDV (SEQ ID NO:6), KFHFVRGSPFGMDV (SEQ ID NO:7), or KFRFVSGSPFGMDV (SEQ ID NO:8).

4. The antibody of claim 1, wherein the light chain CDR1 is RASQSVSSYLA (SEQ ID NO:10), RASQSVSSYLS (SEQ ID NO:11), RASQSVSSYMA (SEQ ID NO:12), or RASQSVSSYLE (SEQ ID NO:13).

5. The antibody of claim 1, wherein the light chain CDR2 is DASNRAT (SEQ ID NO:15), DASNRAP (SEQ ID NO:16), DASNRAM (SEQ ID NO:17), or DASNRAE (SEQ ID NO:18).

6. The antibody of claim 1, wherein the light chain CDR3 is QQRSNWPT (SEQ ID NO:20) or QQRANWPT (SEQ ID NO:21).

7. The antibody of claim 1, wherein the antibody comprises the same heavy chain CDR1-CDR3 and/or the same light chain CDR1-CDR3 as those of antibody 12A4a, 12A4b, 12A4c, 12A4d, 12A4e, 12A4f, 12A4g, 12A4h, 12A4i, 12A4ad, 12A4ba, 12A4bd, or 12A4bad.

8. The antibody of claim 7, wherein the antibody comprises the same heavy chain variable region and/or the same light chain variable region as those of antibody 12A4a, 12A4b, 12A4c, 12A4d, 12A4e, 12A4f, 12A4g, 12A4h, 12A4i, 12A4ad, 12A4ba, 12A4bd, or 12A4bad.

9. The antibody of claim 1, wherein the antibody is a full-length antibody or an antigen binding fragment thereof.

10. The antibody of claim 1, wherein the antibody is a single-chain variable fragment (scFv).

11. The antibody of claim 1, wherein the antibody is a human antibody or a humanized antibody.

12. A nucleic acid or a set of nucleic acids, which collectively encode an antibody that binds human PDL1, wherein the antibody is set forth in claim 1.

13. The nucleic acid or the set of nucleic acids of claim 12, which is a vector or a set of vectors.

14. The nucleic acid or the set of nucleic acids of claim 13, wherein the vector(s) is an expression vector(s).

15. A host cell comprising the nucleic acid or the set of nucleic acids of claim 11.

16. The host cell of claim 15, wherein the host cell is a bacterial cell, a yeast cell, an insect cell, or a mammalian cell.

17. A pharmaceutical composition comprising an antibody of claim 1 or a nucleic acid or set of nucleic acids encoding the antibody, and a pharmaceutically acceptable carrier.

18. A method for inhibiting PDL1-positive cells, comprising administering to a subject in need thereof an effective amount of the pharmaceutical composition of claim 17.

19. The method of claim 18, wherein the subject is a human patient having cancer.

20. A method for producing an antibody binding to human PDL1, the method comprising:
(i) culturing a host cell of claim 15 under conditions allowing for expression of the antibody; and
(ii) harvesting the antibody thus produced.
